(19) United States
(12) Patent Application Publication
(10) Pub. No.: US 2024/0181270 A1
(43) Pub. Date: Jun. 6, 2024

(54) SYSTEM AND METHOD FOR AN ELECTROMAGNETIC FIELD HOLISTIC HEALTH AND SEXUAL WELLNESS DEVICE

(71) Applicant: OSexiFi,. Inc., Dover, DE (US)

(72) Inventor: Balkees Abderrahman, Houston, TX (US)

(21) Appl. No.: 18/531,680

(22) Filed: Dec. 6, 2023

Related U.S. Application Data
(60) Provisional application No. 63/430,573, filed on Dec. 6, 2022.

(51) Int. Cl. A61N 2/00; A61N 2/02

(52) U.S. Cl. CPC A61N 2/004; A61N 2/02

(57) ABSTRACT

A personal stimulation device for holistic health and sexual pleasure, utilizing a mechanical vibration stimulation element and a crystalline core to amplify and focus PEMF generated pulsating electromagnetic fields to stimulate biological tissue, with the electromagnetic fields formed via a coil wrapped around the crystalline core.

### PRIORITY CLAIM

This non-provisional utility patent application claims the benefit and priority to U.S. provisional application 63/430,573, filed Dec. 6, 2022. The above referenced application is incorporated as if restated in full.

### TECHNICAL FIELD

This invention generally relates to the field of holistic health and sexual wellness devices, and more specifically to a new and useful system and method, with mathematical (geometric and harmonic) wave-engineering, for a pulsed electromagnetic field holistic health and sexual pleasure device.

### BACKGROUND

At least 50% of men and 60% of women worldwide suffer from sexual dysfunction. At least 80% of women worldwide do not orgasm from intercourse alone versus 90% of men that do. Around 75% of women (over 65 years) suffer from urinary incontinence (due to weak pelvic floor muscles from aging, entering menopause, and giving multiple vaginal births). At least a ⅓ of women experience vaginal prolapse (due to weak pelvic floor muscles). At least 80% of men (over 70 years) suffer from benign prostatic hyperplasia (BPH). Roughly 52% of men experience one form of erectile dysfunction (ED). Around 30-40% of men experience premature ejaculation (PE). And over 1 millions cases of sexually-transmitted diseases (STDs)/day occur worldwide, 374 million cases/year, according to the World Health Organization (WHO).

Existing sex therapy is expensive and is not covered by most insurance plans (e.g., 10 tablets of Viagra® (50 mg) cost at least $700, taking into consideration that they do not work as well in women as they do in men). Counselling for an individual (45-60 minutes) costs roughly $120-$180 whilst for a couple (75-90 minutes) $190-$310. Vaginal rejuvenation surgery costs at least $4,750 whilst penis enlargement surgery around $15,000 (taking into consideration the adverse effects, commuting to and from hospital, and inpatient and/or outpatient cost until recovery).

At least 30,000 scientific and medical studies corroborate the significant wellness benefits of PEMF, including the successful use of PEMF for peripheral nerve stimulation, and the significant improvements in the sexual functions of vulva and vagina-bodied and scrotum and penis-bodied individuals. One PEMF clinical trial (in vulva and vagina-bodied individuals) reported: “Significant objective and subjective improvements: reducing vaginal laxity, strengthening pelvic floor muscles, and improving female sexual functions (of desire, arousal, lubrication, satisfaction, orgasm, and reduced pain/discomfort), for at least 12 weeks post procedures, with confirmed histological improvements, and none-mild side effects”. Another PEMF clinical trial (in scrotum and penis-bodied individuals) reported: “Significant improvements in erectile dysfunction, premature ejaculation, dissatisfaction, and infrequency, compared to baseline”. Yet another clinical trial (in couples of vulva and vagina-bodied and scrotum and penis-bodied individuals) reported: “Significant improvements in multiple sexual dimensions in both partners”.

American-Serbian electrical engineer and inventor, Nikola Tesla, invented the Tesla (PEMF) coil, and championed the use of PEMF for health and wellness (medical diathermy). This aligns with Dr. Jerry Tennant's assessment, in his book Healing Is Voltage: “Every cell in the body is designed to run at −20 to −25 millivolts. To heal, we must make new cells. To make a new cell requires −50 millivolts. Chronic disease occurs when voltage drops below −20 and/or you cannot achieve −50 millivolts to make new cells. Thus, chronic disease is always defined by having low voltage.”

The NASA Johnson Space Center uses PEMF to maintain the health of astronauts in orbit, and has rigorously researched the use of PEMF in human subjects, including a 4-year study on the use of PEMF for nerve growth and repair: “Physiological and Molecular Genetic Effects of Time-Varying Electromagnetic Fields on Human Neuronal Cells”. NASA's studies concluded that PEMF improves cell vitality, function, repair, and growth.

Scientific and medical research show that harmonic vibrations (i.e., musical frequencies) (FIG. 6A-B), mathematically-constructed with consonant whole-numbered ratio relationships to the fundamental frequency (FIG. 11A-B), evoke significant physiological and psychological health benefits. Harmonic frequencies can be seen in the Pythagorean Harmonic Scale (C D E F G A B C′=tonic, 2^{nd}, 3^{rd}, 4^{th}, 5^{th}, 6^{th}, 7^{th}, unison); Ancient Harmonic Scale (C D E F#G A Bb B C′=tonic, 2^{nd}, 3^{rd}, 4^{th} augmented, 5^{th}, 6^{th}, 7^{th} minor, 7^{th}, unison); and Modern Harmonic Scale (PQRSTUVW P′=tonic, 2^{nd}, 3^{rd}, 4^{th} augmented, 5^{th}, 6^{th} minor, 7^{th} minor, 7^{th}, unison).

Quantum mechanics research shows that the mathematically-constructed spatial components of an electromagnetic system (i.e., geometric and harmonic designs) yield significant efficacy and power benefits. Geometric and harmonic electromagnetic system engineering is seen across the natural world, from the quantum scale (electrons, positrons, protons) (FIG. 8A-B), to that of the astronomical (planets, stars, black holes) (FIG. 8D), and was shown to resonate with the electromagnetic fields of the human body (FIG. 2, FIG. 3A-B, and FIG. 8C).

First, geometric and harmonic wave-engineering designs underpin the perfectly-ordered and functioning, natural and cosmic systems, which inspired breakthrough innovations in science; art/architecture; and economy (FIG. 7A-C). This includes using integers with ratio (logos) and proportion (analogia), as seen with the Fibonacci and Lucas sequences that govern all growth and diminution natural phenomena, and the irrational numbers associated with the Platonic Solids (Pi [π, 3.141], Phi [Φ, 1.618], Phee [φ, 0.618], Euler [e, 2.718], Feigenbaum constant (δ, 4.669), √2, √3, and √5) that govern all growth and diminution natural phenomena, as well as, EM and gravity (FIG. 4A-C, FIG. 5A-D, FIG. 8A-D, FIG. 9A-C, FIG. 10A-D, and FIG. 12A-C). The “Golden Section, Golden Mean, Golden Ratio, Divine Proportion” is actually the “Arithmetic Mean” of the “Geometric Mean” of the two 3-termed continuous geometric proportions (1:3:3:9 and 1:2:2:4 at the Lambda's pyramidion=(√1*3+√1*2)/2=π/2=Φ(^{˜}1.6) with Φ being the invariant, repeating ratio). The “Harmonic Mean” of the Lambda's pyramidion generates some of the most harmonic musical intervals, which are the 5^{th} and 4^{th} (√(2*1*3)/(1+3)=3:2 and √(2*1*2)/(1+2)=4:3). The 2 numbers of the Lambda's pyramidion form the Fibonacci (after Leonardo of Pisa or Fibonacci), Lucas (after François Lucas), and Balkees (after Balkees Abderrahman) sequences, which are unique geometric progressions that maintain Φ or ϕ, and underpin all natural phenomena. Lambda is based on extended continuous geometric proportion, which is seen throughout the natural world (1:2:2:4:4:8 [1:2 ratio] and 1:3:3:9:9:27 [1:3 ratio]).

The Fibonacci ([1, 2], 3, 5, 8, 13, 21, 34, 55, 89, 144, 233, 377 . . . ), Lucas ([2, 1], 3, 4, 7, 11, 18, 29, 47, 76, 123, 199, 322 . . . ), and Balkees ([2, 3], 5, 8, 13, 21, 34, 55, 89, 144, 233, 377 . . . ) sequences are pervasive in the cosmos and have inspired breakthrough innovations:

In the family trees of bees and rabbits (1, 1, 2, 3, 5, 8, 13 . . . ).

In the horn plates of turtles (13 with 5 centered, 8 on the edges, and 5 paw pins).

In the vertebrae of Gabon snake (144).

In the teeth of hyenas (34) and dolphins (233).

In the extremities (5 parts/each extremity), belly (8 segments), and legs (8) of spiders.

In the clouds of hurricanes.

In the DNA nucleotides (34 angstroms long by 21 angstroms wide, for each full cycle of its double helix spiral, with 34:21=Φ).

In uranium oxide (U₂O₅, U₃O₈, U₅O₁₃, U₈O₂₁, U₁₃O₃₄).

In the periodic table's chart of nuclides (neutron/proton number and the atomic number/neutron=Φ).

In the atomic structure arrangement (in a golden spiral with a divergence angle of 4π/(2n−1), and the atomic radius of hydrogen in methane (Bohr radius over Φ). Also, L. Hardy's work on the probability of entanglement for two particles projected in tandem showed Φ over negative 5.

In the bond order between atoms (increasing bond order corresponds to a divergence angle of π/2 on a golden spiral), and the nature of electrons (as seen through electron diffraction), as well as, protons being wave structures (with extranuclear electrons constituting a single spherical “standing wave” that commensurates with a logarithmic optimization pattern).

In the spiral patterns of phyllotaxis (parastichies with 13:8 spiral, some with 34:21 spiral, and others with a divergence rotation golden angle [137.5° ]-driven spiral), sunflower head [55:34, 89:55], daisy petals, pine cone scales [5:3, 8:5], artichoke scales [8 spirals one direction:5 spirals the other], pineapple scales [21 steep: 13 medium:8 gradual, aspiring towards Φ² or Φ+1], pussy willow flower [13 buds:5 turns], cacti areoles, and monkey puzzle tree [13:8].

In the patterns of stock market (B. de Groot's work on Φ in relation to the economic cycles of GDP growth and R. Elliott's “Elliott Wave Theory” on Φ in relation to the recurring fractal wave patterns in stock price movements and consumer behavior, FIG. 7A).

In the patterns of “game theory” (R. Suleiman's work “aspirations-homeostasis theory”), computational electromagnetism, and ethical cognition.

In harmonic music (octave [2:1], fifth [3:2], major and minor sixths [5:3 and 8:5], the piano's 5 black notes; 8 white notes; and 13 notes spanning an octave).

The Lucas sequence and/or angle (360°/(1+)Φ2)=99.5° is found in certain cedars, sequoias, balsam tress, and phyllotactic plants. The Fibonacci, Lucas, and Balkees sequences are bio-serving-and-enhancing mathematical models and geometries, which form the basis of this invention. As few examples, science has shown that the Φ-based geometric arrangement:

Of leaves maximizes photosynthesis (sunlight, rain, and insect pollination exposure of each and every leaf), and in roots maximizes moisture exposure.

Of sunflower seeds maximizes their number in a reduced space (i.e., packing efficiency).

Of a hawk's spiral flight ensures aerodynamic efficiency (i.e., maintaining eye contact with prey without turning its head).

Of the human ear's cochlear spiral enhances the mechanical response to low frequencies and minimizes the cochlear space occupancy within the skull (i.e., anthropometry for packaging efficiency).

Of the human heart's artery branches maximizes vascularization per volume (even in the event of atherosclerotic blockages in other branches) and to the body. The mean ratio between the duration of the diastole and that of the systole is Φ (also the mean ratio between R-R/diastole duration and that of exhalation:inhalation is Φ).

Of the human upper limb's partitions (hand, forearm, and arm) maximizes limb functionality and use (FIG. 8C). Also, the hand's partitions (phalangeal functional lengths being in 1:1.3:2.3 proportions for the first four fingers, and 1:1:2 for the little finger) maximizes hand grip flexibility (despite the rigid phalanges). The 3 bones of each hand's finger are in Φ, and the wrist divides the hand and forearm at the Golden Section.

Of the human mouth's teeth (13 in each quarter of one's mouth over a lifetime: 5 childhood and 8 adulthood) maximizes the packing efficiency and thus mastication power.

Of the human gate phases (durations of stride and stance, stance and swing, and swing and sum of the two double phases are in the Φ ratio, FIG. 8C). This facilitates locomotion control and minimizes energy consumption (while walking and running). It also inspired engineering innovations in robotics.

Of the DNA nucleotides simplifies the codification of complex information (through using a single repeating pattern for different scale levels; what's known as “fractals, autosimilarity, gnomon”).

Second, geometric and harmonic wave-engineering designs, underpin matter formation, through “standing wave” formation (i.e., rejuvenative effect of PEMF in enhancing the biochemical signaling pathways, including building essential proteins that are lost with aging and disease). Standing waves, which form matter, occur only with geometric PEMF and harmonic frequencies (including resonance due to interference between waves reflected back and forth at the resonator's resonant frequency).

Last, scientific and medical studies have shown that human subjects always choose the geometry with the golden ratio over that without proportion, and that the golden geometry resonates with the human eyes and nervous system. As one example, the golden rectangle approximates the human visual field (called the “perimetric hypothesis”). As another, exposure to geometry with the Golden Ratio activates a set of cortical neurons (lateral occipital gyrus for object recognition, and precuneus and prefrontal areas for visuospatial imagery; episodic memory retrieval; and self-processing operations that intelligently regulate humans' thoughts, actions, and emotions through extensive connections with other brain regions), as well as, the amygdala (emotions and memories), and right insula (emotional, cognitive, and motivational functions).

Vulva-and-vagina-bodied individuals have a sex organ, which is anatomically-made of a vulva, clitoris, vagina, cervix, uterus, ovaries, and perineum (anal sphincter muscle, urogenital diaphragm, and pelvic diaphragm). The clitoris (“glans” with 8,000 nerve endings, “body” with erectile tissue, “bulbs” with cavernous nerves and erectile tissue, and “crura” with cavernous nerves and erectile tissue, FIG. 1B; FIG. 3A; and FIG. 5A) is the epicenter of such sex organ, with its major nerve type being A-beta (responsive to touch and vibration with pleasurable sensations) and minor nerve types being A-delta and C (communicates temperature changes and pain sensations). Scientific and medical research has confirmed that the clitoris is chiefly-engineered for sexual pleasure and gynecological benefits, as follows:

Sexual nerve stimulation was shown to convey anti-depressant, anxiolytic, sedative-analgesic, cardiovascular system, anti-cancer, immune system, and longevity benefits. American psychologist, Barry R. Komisaruk, popularized sexual nerve stimulation to reduce the pain threshold (especially during vaginal birth) and improve overall wellness.

Sexual nerve stimulation contracts the two muscles (ischiocavernosus and bulbospongiosus) that surround the vaginal canal, through a lower spinal reflex arc (intact even in individuals with upper spinal cord injuries) (FIG. 1B). This sexual nerve-driven muscle stimulation contributes to easier childbirths in younger women, and prevents urinary incontinence in women as they age; enter menopause; and undergo vaginal births. American gynecologist, Arnold Kegel, invented the Kegel perineometer (exercises), as a non-surgical treatment of urinary incontinence from pelvic floor muscle weakness and/or laxity, through exercising the ischiocavernosus and bulbospongiosus muscles.

Sexual nerve stimulation (chiefly the cavernous nerves, FIG. 3A) contributes to clitoral erection, and vaginal lubrication and lengthening, for self and couple pleasurable intercourse. The pressure generated on both sides of the vagina and urethra, due to clitoral erection (bulbs and crura swelling having been filled with blood), enhances the pleasure sensation; promotes vaginal lubrication (with the help of the feminine hormone estrogen); and vascularizes the area with fresh oxygenated blood. This deeper clitoral stimulation (of the cavernous nerves surrounding the body, bulbs, and crura) can help women with genital mutilation (200 million women worldwide, according to the WHO).

Sexual nerve stimulation (chiefly the lower end of the vaginal canal and the front wall of the vagina, with a higher density of nerve endings, and where the clitoris comes in direct contact with the vagina) activates the: a) Bartholin glands (greater vestibular glands, FIG. 1B) that secrete mucus to lubricate the vagina upon sexual stimulation; b) perineal sponge (blood vessel network) that swells upon sexual stimulation and narrows the vaginal opening for pleasurable intercourse; and c) G-spot is (blood vessel and nerve network, located between the front side of the vaginal wall and back side of the urethral canal, about 0.5-3 inches inside from the vaginal opening) that swells upon sexual stimulation and contributes to the sensation of pleasure. The G-spot is named after German physician, Ernst Gräfenberg. A scrotum-and-penis bodied individual also has a G-spot (P-spot, ^{˜}2 inches inside the rectum), whose stimulation can produce powerful orgasms and contribute to generating thinner seminal fluid from the prostate (for better sperm motility, stability, and energy, thus improving fertility and conception).

Sexual nerve stimulation (chiefly the upper end of the vaginal canal, with a lower density of nerve endings, and where the clitoris does not come in as much contact with the vagina as the lower end) activates the: a) A-spot nerve network that is located on the front side of the vaginal wall (just behind the bladder); b) O-spot nerve network that is located between the back side of the vaginal wall and front side of the rectum (just under the cervix); and c) cervix (with sufficient sexual stimulation leading to the physiological change of titling the uterus forward towards the belly button, which elongates the cervix, allowing for deeper pleasurable penetration, and potentially powerful cervical orgasms with pleasurable uterine contractions).

The physiology of the orgasm (in vulva and vagina-bodied and scrotum and penis-bodied individuals) reflects the mechanics of recoil. Recoil is the result of “conservation of momentum” per “Newton's third law”: the force required to accelerate something will evoke an equal but opposite reactional force; meaning the forward momentum gained by sexual nerve stimulation and muscle contraction via the spinal arc, will be mathematically-balanced out by an equal and opposite momentum exerted back with erection (pressure) and/or ejaculation (projectile). Accordingly, the orgasm recirculates the nerve signals and blood flow, thus rejuvenates the sex organ through “kinetic energy” within the clitoris; onto adjacent pelvic organs; and up the central nervous system, in alignment with the law of “conservation of energy”. This orgasm-driven, clitoral, kinetic energy aligns with the “Hamiltonian” of a system (i.e., quantum mechanics), which corresponds to the total energy of that system, including both kinetic energy and potential energy (i.e., energy held by a system because of its position relative to other systems, as seen through the clitoris's strategic anatomical connection to adjacent pelvic organs, through its suspensory ligament; pars intermedia; albuginea; ischiocavernous and bulbospongiosus muscles; and Kobelt's plexus). According to the “Hamiltonian” of a system, the system's energy spectrum is the set of possible outcomes obtainable from a measurement of the system's total energy, per the “time-evolution” of a system, as seen with the “sexual response cycle” over time. This explains the myriad, medically-proven benefits of the phases of the “sexual response cycle” (including orgasms).

The two “crura” of the clitoris are made of two “corpora cavernosa”, which are erectile tissue cavities that fill with blood upon sexual excitation and are covered with a fibrous envelope (albuginea). They adhere strongly to the inner face of the ischio-pubic branches of the pelvic bones (while being covered by two ischiocavernous muscles). The contraction of these muscles pushes the blood from the “crura” into the “body”, leading to clitoral erection.

The two “bulbs” are made of two “corpus spongiosum”, which are erectile tissue cavities that fill up with blood upon sexual excitation and are not covered with the albuginea. They communicate with the “body” of the clitoris via a network of veins (Kobelt's plexus), descend on either side of the urethra and the vagina, and are covered by two bulbospongiosus muscles (inserted backwards at the level of the transverse muscles of the perineum and join together forwards at the level of the angle of the clitoris). The contraction of these muscles, compresses the dorsal vein of the clitoris (via a band they form at the back of the angle), and pushes the blood from the bulbs into the body, thus enhancing the clitoral erection.

The “body” of the clitoris is formed by the union of the two corpora cavernosa, which are separated from each other by an incomplete medial septum, all of which is enveloped in the albuginea. The proximal part of the body is first directed forwards and upwards (in the same plane as the crura), then bends to form the angle of the clitoris, and the distal part of the body (shaft) moves downwards and backwards. There lies a ligament that keeps the clitoral body suspended from the pubic symphysis. Upon erection, this angle does not widen, instead the body of the clitoris increases in volume and tightens, as part of the “recoil” mechanics.

“Pars intermedia” is a pyramidal-shaped area (FIG. 5A), which contains the Kobelt's venous plexus that connects the bulbs to the body, and establishes a vascular connection with the vessels of the vestibule walls that communicate with those of the labia minora.

The “glans” is a pyramidal-shaped area (FIG. 5A), chiefly made of connective tissue and some erectile tissue, lying adjacent to the albuginea (on its inner surface), and attaches to the labia minora (by the skin fold “frenulum”).

Embryology research shows that the feminine clitoris is the homologue of the masculine penis (i.e., both are from the same embryonic components but develop into a different size and shape during pregnancy, FIG. 1A). Therefore, the invention's optimized (geometric and harmonic) electromagnetic fields can resonate with and benefit both the vulva and vagina-bodied and scrotum and penis-bodied individuals, making it a sex/gender-inclusive bio-integrated system.

Scientific research (including that of X-ray diffraction) shows that crystals make the closest approximation to the mathematical purity of regular solids, making their transparent, molecularly-ordered (geometric) lattice with its refractive index, a PEMF conductor; polarizer; and amplifier (FIG. 12 and FIG. 13). The molecules of crystals are lined up in tens, even hundreds of millions, of identical molecules, in unit-cell arrangements of one or more of the 14-Bravais lattices, which enables the molecules to repeat orderly and indefinitely (i.e., “space group” in mathematics, physics, and chemistry; “symmetry group” in group theory; “Bravais lattice” in geometry and crystallography; “Archimedean symmetries and 3D tessellations” in geometry), which enhances the invention's PEMF conduction.

Double refraction is an optical property of a distinct type of anisotropic, uni or biaxial, negatively or positively-birefringent crystals, discovered by Erasmus Bartholinus (i.e., a single beam of unpolarized electromagnetic waves (light) interacts with the local electrical components of the crystalline lattice and, as a result, splits into two linearly-polarized beams: ordinary ray (O-ray) that obeys Snell' Law; propagating at constant velocity and in a spherical wavefront based on the Huygens' principle of wavelets emanating from a point source of light in a medium (FIG. 12A-B), and extra-ordinary ray (E-ray) that does not obey Snell' Law; propagating at varied velocity and in an ellipsoid of revolution (FIG. 12A-B), both of which have planes of polarization that are mutually-orthogonal (perpendicular) to each other). Negatively-birefringent crystals have O-rays with slower velocity [higher effective refractive index] and E-rays with faster velocity [lower effective refractive index], whereas, positively-birefringent ones have the opposite.

Double refraction is based on the laws of electromagnetism, first proposed by British mathematician and physicist James Maxwell (i.e., velocity of light through a material=speed of light in a vacuum (c) divided by the product of the square root of the material's dielectric constant (e) multiplied by the magnetic permeability (m) of the material).

Gynecology (IVF) takes advantage of the birefringence characteristics in sperm heads (to select spermatozoa for intracytoplasmic sperm injection) and in oocytes (to select the ones with highest chances for successful pregnancy). This reflects the crystalline and EM nature of sperms and oocytes, which is aligned with the invention's engineering design (FIG. 3A).

Crystals are also primed to interact with the optical, acoustical, thermal, magnetic, and electrical events surrounding them, making this invention an ideal candidate for a bio-integrated system that improves the electromagnetic fields of the human body (including those of the sex organs).

Given that the existing PEMF wellness, gynecological, and sexual pleasure devices are non-optimized mathematically; that the existing sexual pleasure devices do not use PEMF; and the world population continues to experience sexual dysfunction alongside myriad wellness challenges, there is a need for a new, useful, effective, safe, user-friendly, commercial, sex/gender-inclusive, holistic health and sexual pleasure PEMF and/or other wave system and method.

### SUMMARY

The invention is carefully-engineered taking into account what follows:

How the thickness of the crystalline lattice impacts its refractive index (i.e., optical path difference D=(refractive index 1 [n1]−refractive index 2 [n2])*crystal thickness [t], and retardation (r)=thickness (t)×birefringence (B)).

How the dielectric constant of a crystal impacts the speed at which incident PEMF propagates through it (i.e., crystallographically-distinct axes of a crystal interact with incident PEMF by a mechanism dependent upon the inherent orientation of the lattice's electrical vectors and the direction of the wave's electric vector component).

How the refractive index of a crystal varies with the direction of incident PEMF (i.e., birefringence=E-ray refractive index n(e)−O-ray refractive index n(o)), making PEMF's geometric coil design around the crystalline core of supreme importance, to reduce PEMF retardation;

generate the co-axial trajectories of the O-rays and E-rays thus generating radial polarization (i.e., electric field vector points towards the center of the beam, focusing the invention's PEMF beam over the targeted anatomy, FIG. 12B); and preserve the PEMF field intensity per Malus' law (I=I₀ cos² θ; θ is the angle between the direction of polarization of incident PEMF and the polarization axis of the polarizer).

How the optical transparency of a crystal impacts its dielectric permittivity tensor and thus ability to become circularly-birefringent (“Faraday effect” or “magneto-optic Faraday effect (MOFE)”, under the influence of magnetic fields, whereby polarization rotation is proportional to the projection of the magnetic field along the direction of PEMF propagation, which further enhances the PEMF beam).

How the combination of a crystal and PEMF generates the “Kerr effect” and “Pockels effect” (i.e., “Kerr effect” or the “quadratic electro-optic (QEO) effect” is when the refractive index of a material is changed in response to an applied electric field, whereas, the Pockels effect is when the induced change in refractive index is directly-proportional to the square of the electric field, instead of varying linearly with it, which causes changes in birefringence that vary in proportion with the strength of the applied electric field).

How the birefringence of a crystal plays an important role in “second-harmonic generation”: frequency doubling (i.e., twice the frequency and half the wavelength; by the musical interval octave), “sum-frequency generation”, and “harmonic generation”, which conserve the coherence of the excitation, and further enhances the PEMF beam.

How the geometric and harmonic combination of a crystal and PEMF optimizes the “photoelectric effect” (i.e., emission of electrons “photoelectrons” when EM radiation hits a material, as an effect of waves interacting with each other), and reduces the “work function” (i.e., electron and energy scattering).

How the geometric and harmonic combination of a crystal and PEMF generates “standing waves” (i.e., waves oscillating back and forth that create “interference patterns”, which produces the electron/positron pair, and transforms “free-state” photons to “confined-state” photons. “Confined photons” are patterns of integer-wavelength loops that create charged particles. Charged particles rotate to form atoms, atoms combine to form molecules, and molecules combine to create matter).

How the geometric parameters of the coil surrounding the crystalline core can generate purer and more robust PEMF fields (e.g., handedness (chirality), dilation symmetry, number of turns, turn spacing (minor and major grooves, if double helix), knotting patterns with electrical charge differences, packing density, radius of the helical cylinder, helical pitch angle, helical tube radius, coil diameter, coil n-based repetition length that defines the slope of the curve, and number of winds wrapped around a torus of major radius R and minor radius r with t based on the number of sets of xyz coordinates).

### BRIEF DESCRIPTION OF THE FIGURES

FIG. 1 is a schematic representation of the invention's anatomy-based engineering. (A) homologous, embryonic, internal and external genitalia for vulva and vagina-bodied and scrotum and penis-bodied individuals. (B) homologous, adult, internal and external genitalia for vulva and vagina-bodied and scrotum and penis-bodied individuals.

FIG. 2 is a schematic representation of the invention's nervous system-based engineering. Nervous system-based electromagnetic field mapping of the human body.

FIG. 3 is a schematic representation of the invention's nervous system-based engineering. (A) Nervous system-based electromagnetic field mapping of the sex organs of vulva and vagina-bodied and scrotum and penis-bodied individuals. (B) Reflexology science-based polarity mapping of the human body (including sex organs).

FIG. 4 is a schematic representation of the mathematical concept and geometric representations of the “Golden Section” used in the invention. (A) Lambda/Tetraktys-derived geometric progressions that maintain Φ or ¢ (Fibonacci sequence uses [1 and 2], Lucas [2 and 1], and Balkees [2 and 3 as the union of the first even female number 2 (also 2^{nd} triangular number) and the first odd male number 3 (also 3^{rd} triangular and 2^{nd} tetrahedral number)). The Lucas integer sequence is uniquely formed by alternately adding and subtracting the golden powers of Φ and its reciprocal 1/Φ, whereas, the Fibonacci and Balkees sequences from adding the golden powers of Φ and its reciprocal 1/Φ. (B) “Golden Section” of a line is when the ratio of the shorter:longer segment=the ratio of the longer:whole, and when the ratio of the whole:longer=longer:shorter segment, which ensures continuous geometric progression. Unity (1) can act as the greater (whole), lesser (shorter), and mean (longer). The golden rectangle, pentagon, pentagram, circle (with golden angle=360°/Φ²=137.5°), and Platonic Solids. Fibonacci, Lucas, and Balkees Golden Series converge on the Golden Ratio Φ or ϕ, with the ratio becoming more accurate as the numbers increase. (C) Fibonacci, Lucas, and Balkees Golden Series underline all cyclical growth and diminution phenomena seen in the natural world. The Fibonacci/Balkees sequences are also seen in the Pascal Triangle (bottom) and the Golden Spiral surrounding the golden triangle (top, right) or within the golden rectangle defined by the Fibonacci/Balkees sequence (top, right). The Fibonacci, Lucas, and Balkees Golden Series underline all natural phenomena because they have the singular, unique ability to grow through fusing multiplication (Gₙ₊₁=Gₙ*Φ) and addition (Gₙ₊₁=Gₙ+Mₙ=Gₙ+Gₙ₋₁), as well as, to shrivel through fusing division (Gₙ₊₁=Gₙ/Φ) and subtraction (Gₙ₋₁=Mₙ=Gₙ−Lₙ=Gₙ−Gₙ₋₂). The fulcrum is Unity, as the geometric mean, in golden relationship to both the increase of the deficient lesser and decrease of the excessive greater. N=number, G=greater, L=lesser, and M=mean. The Golden Spiral (logarithmic, equiangular, growth spiral) is derived from the Fibonacci/Balkees sequences, as well as, the pentagram's arm, which represents gnomonic growth by accretion (i.e., increasing length, width, and size, without varying the proportion, therefore, always relating the parts to the whole, no matter the scale of growth). Such spiral can be derived from the equal divisions of a circle, has the line drawn from its center meeting any part at the same angle for that spiral, is identical at every scale (i.e., fractal, autosimilar, invariant with respect to scale transformation), and is found in leaf spirals; mollusk shells; cacti and seed-head phyllotaxis; whirlpools; hurricanes; and galaxies.

FIG. 5 is a schematic representation of anatomy-based Golden Section-integrated mapping of the sex organs of vulva and vagina-bodied and scrotum and penis-bodied individuals used in the invention. (A) Vulva and vagina-bodied individual's sex organ displays Φ, the golden rectangle, golden triangle, golden spiral, vesica piscis, and pyramidion geometry (specifically the glans clitoris and pars intermedia with Kobelt's venous plexus). The anatomical dimensions are based upon 10 medical and scientific landmark studies. (B) Scrotum and penis-bodied individual's sex organ displays Φ, and (in its erect state) the obelisk geometry. (C) Urogenital and anal triangles approximate the golden gnomon geometry. (D) The female pelvic inlet, cavity, and outlet form triangles (including the 5:12:13 Pythogroean triangle that also applies to lunation and the musical keyboard). The 12 and 13 numbers, as well as, Φ ratios are shared between the female pelvic anatomy, menstrual cycle, lunation, and measures of time and space. Lunations and menstrual cycles form the “pentagon within a circle” geometry; whereby 5 pentagon arms (length 12) accurately generate 60 full moons and menstrual cycles with a 100/Φ ratio, which adds to the scientific evidence showing the impact of the lunar cycle on the female reproductive cycle (menstruation, fertility, and birth rate), in alignment with the human physiology being influenced by seasonal, lunar, and circadian rhythms. This is no different to scientific studies revealing how the lunar cycle affects hormonal levels in insects, reproduction and the hypothalamus-pituitary-gonadal axis in fish, melatonin and corticosterone levels in birds (especially during full-moon days), and taste sensitivity and the ultrastructure of pineal gland cells in rats. Science suggests that the release of melatonin and endogenous steroids, which mediates cyclic alterations of physiological processes, is triggered by PEMF and/or the gravitational pull of the Moon. The 3:11 glans clitoris:female pelvic cavity transverse dimension ratio approximates the 3:11 Moon: Earth size ratio (also found in the Giza pyramids), and generates the geometry of squaring the circle (i.e., perimeter of circle=perimeter of square and area within circle=area within square), as well as, the numbers 33 and 18.618 bound by Φ (i.e., significant calendar numbers, with 33 defining the solar year, and 18.618 (or 18+1/Φ) defining the lunar node period/annual lunation rate; being shared between the eclipse year, solar year, and 13 lunations).

FIG. 6 is a schematic representation of the mathematical geometry in harmonic music used in the invention's harmonic frequencies. (A) Overtones with harmonic intervals between frequencies (first row) and major scale (second row). Enharmonicism and Chromticism combing the circle of fifths with the spiral of fifths to produce all possible spellings for the 12 notes with equal temperament (third row [left]), and the Pythogroean circle of perfect fifths ending with the Pythogroean comma (^{˜}74:73, after 12 fifths/7 octaves, third row [right]). Spirals from repeated progressions of the perfect fourths (4:3), major thirds (5:4), minor thirds (6:5), and major tones (9:8) (fourth row). 12 equally-spaced notes arranged in a circle with the resultant geometry of combined intervals (fifth row). A geometric system of glyphs for musical intervals, with shapes expanding and contracting per the size of the interval (lower part shows the symmetrical nature of consonances, first row [left]). Intervals can be consonant (perfect: tonic/unison, 4^{th}, 5^{th}, octave and imperfect: 3rds and 6ths) or dissonant (2nds and 7ths, need to resolve). A chord is made of 3 musical tones starting with the tonic/root (1:1 frequency and is usually C). Chords can be stable (major/minor) or unstable (diminished/augmented, rootless having no 4ths or 5ths), with shown geometry to reflect such chords (first row [right]). The geometry of musical pitch organization (second row): “modal music” is the most common harmonic system with good variability and softened tone-tendencies, does not need to be major or even be used, and the leading tones can occur in positions other than the standard major/minor. “Tonal music” involves only major/minor (with the dominant cord always major pointing to the tonic with its leading tone), and half steps being highly-organized. “Dronal music” is melodic, and has all scale notes assuming variable roles with interval and repetition that always relates to the central resting tone (i.e., drone). Dodecahedral mapping of the 12 notes of the scale preserving the tritone oppositions in the circle of fifths (third row). (B) Dufay's Vasilissa vocal prelude, ergo gaude, composed with Φ. Russian musicologist Sabaneev discovered that Φ was used in 97% of the compositions of Beethoven, 97% of Haydn's, 95% of Arensky's, 92% of Chopin's, 91% of Schubert's, 91% of Mozart's, and 91% of Scriabin's. Definitions: “musical interval” is a simple ratio between two adjacent frequencies. “Overtones/harmonics” are whole number ratios, higher in frequency (ascending), shortening of a string in wavelength. “Undertones/subharmonics” are whole number ratios, lower in frequency (descending), lengthening of a string in wavelength. A scale is made of musical tones (most commonly 5 or 7. Tones can be chromatic (sharing the alphabet; CC# or EbE) or diatonic (adjacent on the scale, EF); sharp or flat/minor).

FIG. 7 is a schematic representation of the mathematical geometry in optimized economic, artistic, and architectural systems as the basis of the invention. (A) In “chaos theory” (left), Φ governs the chaos border of non-linear systems, whereby order passes into and emerges out of disorder, with the presence of fractal geometry. “Chaos theory” involves viewing dynamical systems (including those of PEMF) as occupying geometrical space (the coordinates of which are derived from the system's variables), and includes “strange attractors” (e.g., Lorenz attractor) and the Feigenbaum constant 4.6692 (δ, δ/π=^{˜}Φ, δ/e=^{˜}Φ, δ−Φ=^{˜}π, δ−π=^{˜}Φ, π+Φ=^{˜}δ, δ/2=^{˜}√5, δ/3=^{˜}Φ, δ/8=^{˜}1/Φ). (B) The Italian Early Renaissance painter Sandro Botticelli's The Birth of Venus (and countless artistic masterpieces) using Φ (including the Rabatment compositional technique). (C) The Greek mathematician and sculptor Phidias's Parthenon standing on the Athenian Acropolis in Greece uses Φ.

FIG. 8 is schematic representation of the mathematical geometry in the quantum (molecular, atomic, subatomic, and Planck), human, and cosmic systems. (A) Geometrical symmetry of the “eight-fold way” seen in octet and decuplet of hadrons (i.e., subatomic particles made of two or more quarks). (B) DNA molecule measures 34 angstroms long by 21 angstroms wide, for each full cycle of its double helix spiral, with 34:21=0 (length:width of the B-DNA molecule=Φ and Major:Minor groove phosphorus atoms=Φ). The rotation of each base pair by 36°, in the canonical B-DNA molecule, gives a complete 360° rotation every 10 base pairs, and results in a decagonal structure and ten-fold rotational symmetry (B, first row). The DNA double helix is modeled as G-ball dodecahedron, resonating up around a central axis (5^{th} musical interval), with harmonic standing wave sharing energy inside Φ-damping (B, second row). B-DNA cross view shows 10 “golden triangles: 72° base angles and 36° vertex angle, sides 1 and base Φ”, and when adding each golden triangle to the abc triangle, 10 “golden diamonds” are formed (sides Φ−1, Φ−1, Φ, Φ), with the axial form of B-DNA being neatly encompassed by these 10 “golden diamonds” with Φ occurrence (B, first row). Plot shows DNA's decagonal super-imposed polygons (e.g., pentagons), when viewed axially, with co-ordinates for the polygon at “6-o'clock” being for the two inner hydrogens of each group of four (−8.4955 Å) and the outer oxygen pair (−10.4890 Å), giving side lengths of 8.9335 and 3.4041 Å, respectively, and generating the ratio of Φ² (B, third row [left]). The DNA nucleotides show the pentagonal geometry with Φ proportions (B, second row [right]). The pentagon geometry is pervasive in life forms from starfish, to the passion flower, and ammonia NH₃; methane CH₄; and water H₂O (with their internal bond angles being 108°=internal angle of the pentagon). (C) The human body with Φ anthropometric proportions (left), including that of stance:swing gait phase (right). (D) The cosmic processes with Φ proportions. Black holes (the point where a black hole's modified heat changes from positive to negative (upper equation), the lower bound on black hole entropy (middle equation), and the loop quantum gravity parameter to black hole entropy (lower equation), are all with Φ proportions) (D, first row [left]). Planets with orbital years in Φ (Mercury, Venus, Jupiter, and Saturn relative to an Earth year, approximates to different powers of Φ) (D, first row [right]). Saturn has its diameter and rings in Φ proportions. The Earth and Moon's dimensions are in Φ ratio as well. Planets (from Mercury to Pluto) have harmonic tunings: audible frequencies (141.27, 221.23, 136.10, 144.72, 183.58, 147.85, 207.36, 211.44, 140.25 Hz), audible tones (D, A, C#, D, F#, D, G#, A, C#), tuning pitches (423.34, 442.46, 432.10, 433.67, 436.62, 443.04, 439.37, 422.87, 445.26 Hz), octave number (30, 32, 32, 33, 36, 37, 39, 40, 40), and wavelengths (0.483 blue, 0.616 orange, 0.501 blue-green, 0.471 blue, 0.743 red, 0.461 blue, 0.685 orange-red, 0.645 orange-red, 0.486 blue). Jupiter and Saturn's synods in Φ (D, second row [left]). Planetary orbits in a golden-spiral (D, second row [right]). The asteroid belt is at a distance t from the Sun, and the inner planets have orbital radii of t/n (orbit of Ceres measuring t and those of the inner planets are rational fractions of Φ, the same pattern was shown to repeat itself for the orbital motion of planetary moons and rings).

FIG. 9 is a schematic representation of the mathematical geometry in the plant and animal kingdoms as the basis of the invention. (A) Egg with Φ proportions. (B) Variety of animals with Φ proportions (e.g., beetles, ladybug, fish, tiger). (C) Spiral phyllotaxis (first row [left]) with 8 leaves:5 turns and the leaves diverging with the golden angle of 137.5°, as well as, frond of brown alga (first row [center]) and sneezewort (first row [right]), with the Fibonacci/Balkees numbers (i.e., in the count of their stems and leaves as they grow). Spiral phyllotaxis (Archimedean or Fermat) with the seed head expanding through forming new primordia at golden angles of 137.5° (second row). The leaves (numerator) are produced in turns (denominator), with the leaf's divergence angle being 137.5°, and as the leaves and turns exponentially growing, the angle further approximates to 137.5° and 222.5° (third row). Spiral phyllotaxis is one of only 3 ways whereby nature arranges leaves along a stem, and is found in 80% of the 250,000 different species of higher plants (the other two are disticious like in corn and decussate like in mint). Leaves not only display Φ proportions to turns, but also within their structure (fourth row).

FIG. 10 is a schematic representation of novel coil engineering based on the mathematical geometry of naturally-occurring optimized PEMF systems. (A) “Archimedean” coiling as a series of parallel, equidistant lines (first row). “Fermat” coiling (or parabolic spiral) as successive whorls that enclose equal increments of area (second and third rows). “Logarithmic” (or growth spiral) with self-similarity and Φ (fourth row). (B) Toroidal helicoidal coiling (from black holes and DNA/molecular knots, all with Φ proportions). (C) Multi-circular coiling (from cell division and embryonic development, all with Φ proportions). (D) Helices symmetrical about an axis (from umbilical cord, microtubules, and DNA, all with Φ proportions). The invention's spiral and/or helical coiling has geometrically-based-and-optimized engineering parameters (e.g., handedness (chirality), dilation symmetry, number of turns, turn spacing (minor and major grooves, if double helix), knotting patterns with electrical charge differences, packing density, radius of the helical cylinder, helical pitch angle, helical tube radius, coil diameter, coil n-based repetition length that defines the slope of the curve). Helical structures have geometrical constraints analogous to the close packing of 3D crystal structures. The invention's toroidal helicoidals has geometrically-based-and-optimized engineering parameters (e.g., number of coil winds wrapped around a torus, of major radius R and minor radius r, with t based on the number of sets of xyz coordinates).

FIG. 11 is a schematic representation of novel harmonic frequency engineering based on number theory and equal temperance. (A) Harmonic frequencies inspired by the works of Pythagoras, Farey, Pascal, Cantor, and Riemann. The overtones (at least 16 ascending harmonic rows) and undertones (at least 16 descending subharmonic columns), with at least 256 intervals in whole-number ratios, perfectly generate the Lissajous figures in ordered musical sequence. (B) The harmonic frequencies' diamond geometry with 4 quadrants that propagate indefinitely. 1: emotional emotive effect, harmonic to the right and subharmonic to the left, with an octave range of 3 upper octaves to the right and 3 lower octaves to the left. 2: spiritual emotive effect, includes only upper harmonics. 3: physical effect, includes only lower subharmonics. 4: includes micro tonalities of both the harmonics and subharmonics, with an octave range of 2 octaves, and can be used for fine-tuning/biofeedback. The 2^{nd} and 3^{rd} quadrants (each) have a range of 4 octaves. Wavelength/frequency mapping of the 4^{th} quadrant perfectly generates the color gradient of the light spectrum. Frequency, interval, and musical notes exemplary mapping of one of the quadrants (i.e., 1st quadrant). This frequency construction is the most harmonic, because it avoids the adjacent tones and half tones being dissonant (as is the case in the current diatonic scale), and allows for key-to-key modulation.

FIG. 12 is a schematic representation of the mathematical geometry and optical properties of crystals used in the invention. (A-B) Double refraction in anisotropic, uni or biaxial, negatively or positively-birefringent crystals. A single beam of unpolarized electromagnetic waves (light), interacts with the local electrical components of the crystalline lattice and, as a result, splits into two linearly-polarized beams: ordinary ray (O-ray) that obeys Snell' Law; propagating at constant velocity and in a spherical wavefront based on the Huygens' principle of wavelets emanating from a point source of light in a medium, and extra-ordinary ray (E-ray) that does not obey Snell' Law; propagating at varied velocity and in an ellipsoid of revolution, both of which have planes of polarization that are mutually-orthogonal (perpendicular) to each other. The diagrammatic ellipsoid (x,y,z) is the “refractive index ellipsoid”, which relates the orientation and relative magnitude of the refractive index in a crystal. Crystals can transmit unpolarized and/or polarized light (i.e., linear, radial, even circular polarization). (C) X-ray diffraction revealing the intensely-ordered inner structures of crystals (C, first row). There are seven crystal systems (triclinic, monoclinic, orthorhombic, tetragonal, trigonal, hexagonal, and cubic) (C, second row). Russian mathematician and crystallographer, Evgraf Federov, deduced at least 230 geometric-groupings for crystals due to their “Bravais lattice, space group, and symmetry group” unique” properties.

FIG. 13 is a schematic representation of the invention's mathematically-based, geometric and harmonic wave-engineering.

FIG. 14 is a schematic representation of an exemplary system architecture that may be used in implementing the system and/or method.

FIG. 15 is a schematic representation of one physical variation of the invention's geometric and harmonic wave stimulation technology. (A) Mathematically-engineered wave stimulation with PEMF (without physical touch) and mechanical vibration (with physical touch). The crystalline lattice (1), insulated coil/supercoil (2), and pulse generator/circuit (3) are mathematically-constructed, per the geometry of the DNA in Φ proportions, to ensure cellular resonance and stimulation. The mechanical vibration motors (4) and crystalline lattice finger grip (5, for ergonomic use and vibration amplification) are mathematically-constructed, per the geometry of the Trinity Celtic knot, which aligns with the anatomy of the sex organ for a vulva and vagina-bodied user (zone 1: glans clitoris and clitoral shaft/body, zone 2: bulbs and crura, and zone 3: vaginal opening, Bartholin glands, perineal sponge, and G-spot), to ensure the distribution and maximization of the sexual and gynecological stimulation, and thus the incurred benefits by a vulva and vagina-bodied user. (B) Mathematically-engineered wave stimulation with PEMF (without physical touch) and mechanical vibration (with physical touch), using an insulated “supercoil” instead of an insulated “coil”, per the geometry of the DNA. The crystalline lattice, coil/supercoil, and pulse generator may constitute 2 parts of the device body, whereas, the mechanical vibration motors and crystalline lattice finger grip may constitute the third part of the device body.

FIG. 16A is an external view of an exemplary embodiment of the device.

FIG. 16B is a cut-away view of an exemplary embodiment of the device.

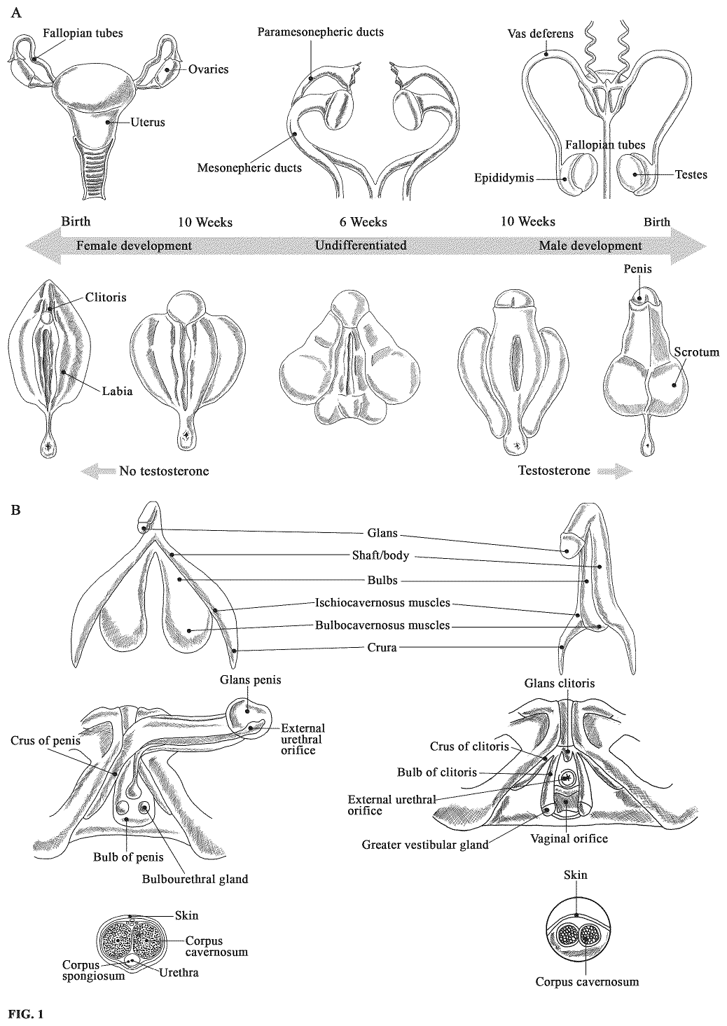

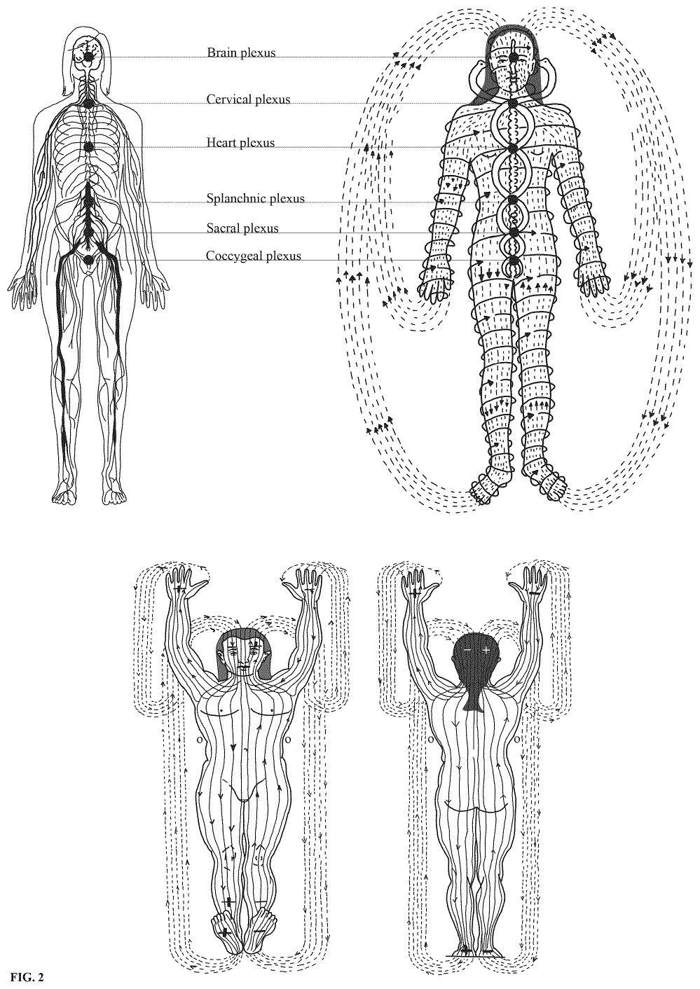

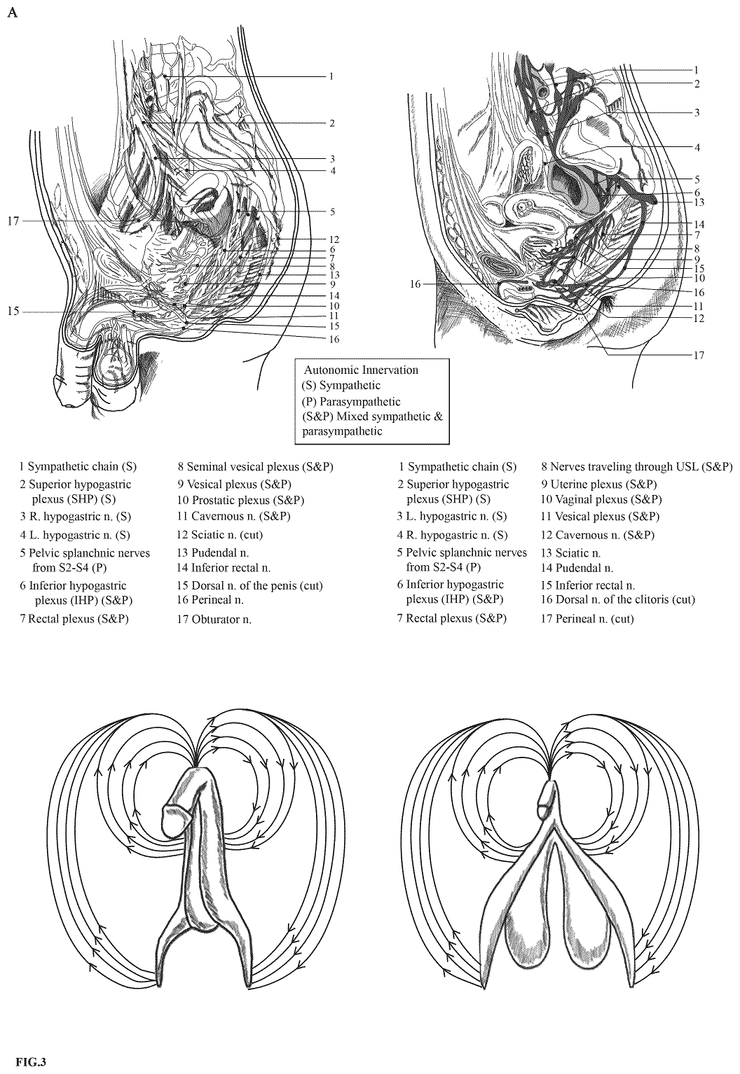

### DESCRIPTION OF THE EMBODIMENTS

The following description of the embodiments of the invention is not intended to limit the invention to these embodiments, but rather to enable a person skilled in the art to make and use this invention.

### Systems and Methods

### Overview

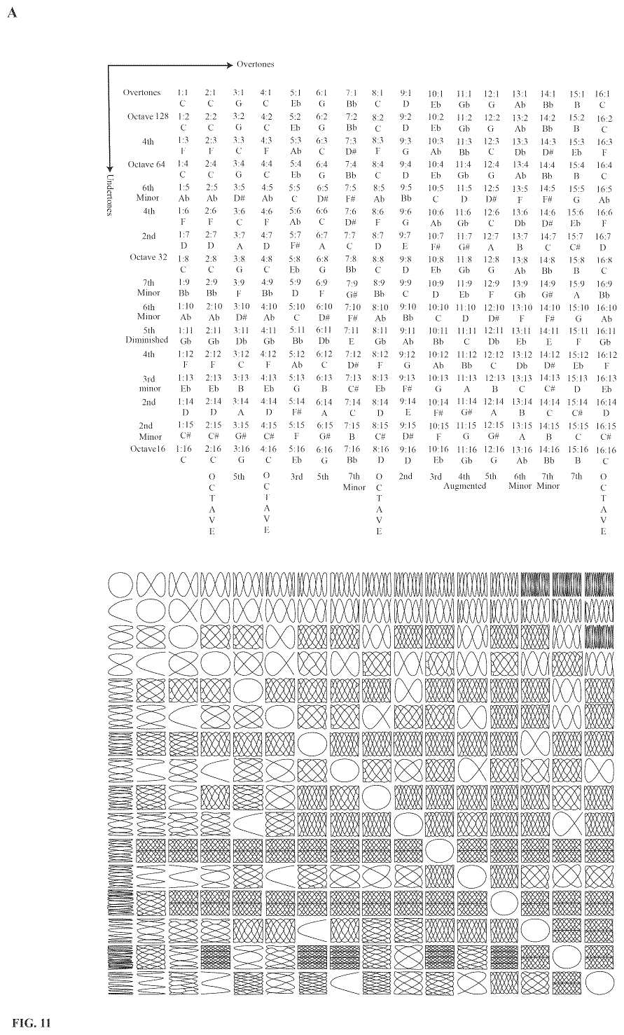

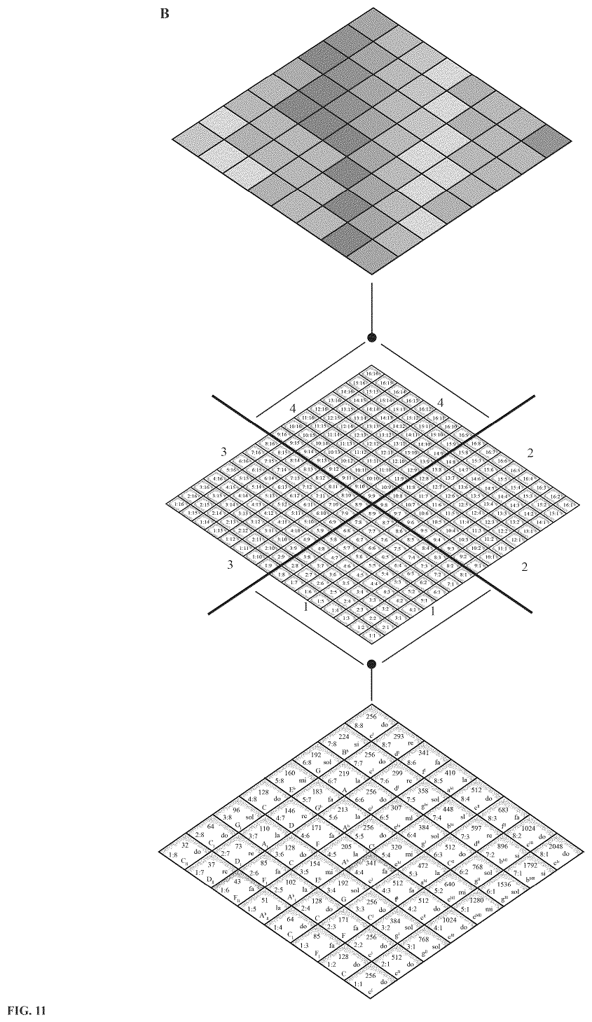

The systems and methods for a PEMF holistic health and sexual pleasure device function to enable a new type of device that enhances a person and/or couple's sexual experience, gynecological health, and overall health and wellness. The systems and methods integrate PEMF and/or other wave stimulators (FIG. 13) with mathematical (geometric and harmonic) wave-engineering designs, into a consumer device. The PEMF and/or other wave stimulators can be used to target specific anatomical regions (sexual wellness) and/or the whole body (holistic health). In particular, the systems and methods can target superficial regions of the sex organ of a vulva and vagina-bodied individual (e.g., clitoral head) and/or deeper regions (e.g., clitoral complex, clitoris-urethra-vagina complex, G-spot, O-spot, A-spot).

### General Description of the Device

The systems and methods preferably use one or more PEMF stimulation elements and/or one or more wave stimulation elements (FIG. 13), integrated into a manipulatable device that can be used by a user, for holistic health and sexual pleasure. Herein, the exemplary stimulation elements of PEMF are used but the system and method are not necessarily limited to only that wave stimulation element.

The systems and methods may be adapted into various form factors and/or include operational capabilities that are customized to the type of user. In one variation, the system and method may be implemented in a device that is intended for vulva and vagina-bodied people. In another variation, the system and method may be implemented in a device that is intended for scrotum and penis-bodied people. In yet another variation, the system and method may be implemented into a device that is non-gendered. Herein, the systems and methods are primarily described and shown as they may be used for a device for vulva and vagina-bodied people. However, the systems and methods may be adapted for various audiences, and for different wellness uses.

The systems and methods may include a variety of the features and elements described herein. In one variation, the system and method may implement PEMF stimulation and/or one or more wave stimulation elements (FIG. 13), under the direct control of a user. In other variations, the systems and methods may include a neuro-biofeedback, neuro-modulation, and/or other feedback technologies, such that the device can learn and then improve; supplement; or enhance a user's experience (e.g., personalizing their PEMF intensity, PEMF frequencies). Variations may also include using voice commands, gesture-based commands, bio-responsive inputs, other types of user-controlled inputs, and/or sensor inputs to operate and control the device.

In one variation, the system and method is implemented to use one or more external stimulation elements (to stimulate from outside the body). In another variation, the system and method may include one or more internal stimulation elements (to stimulate from within the body). In yet another variation, the system and method may include one or more internal and/or external stimulation elements.

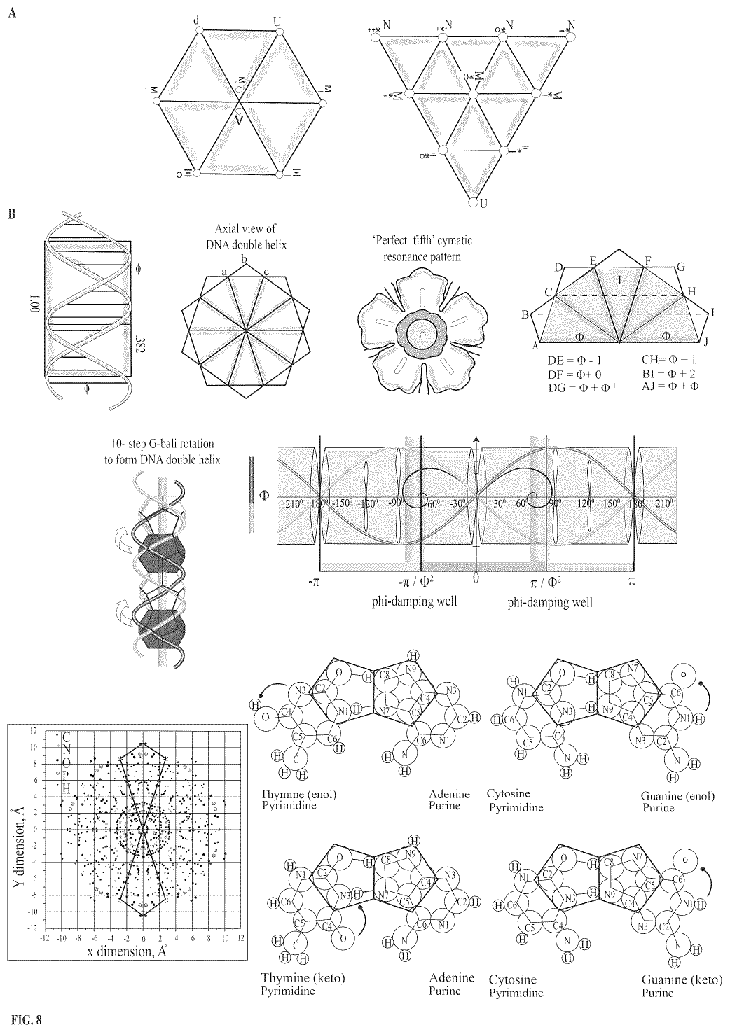

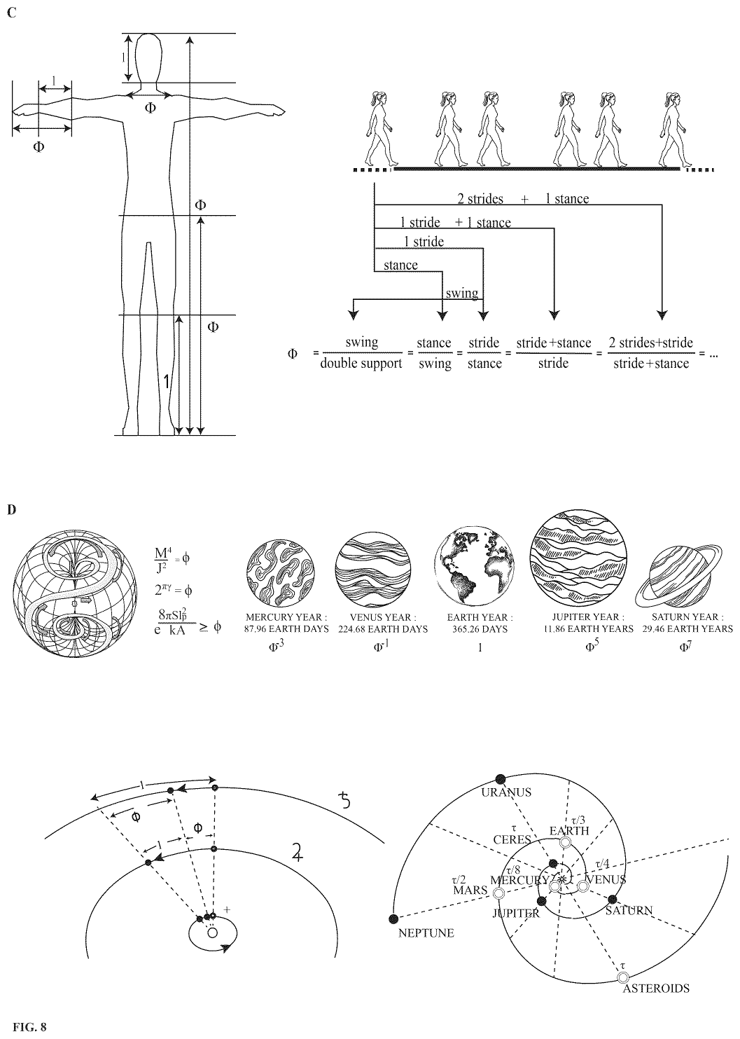

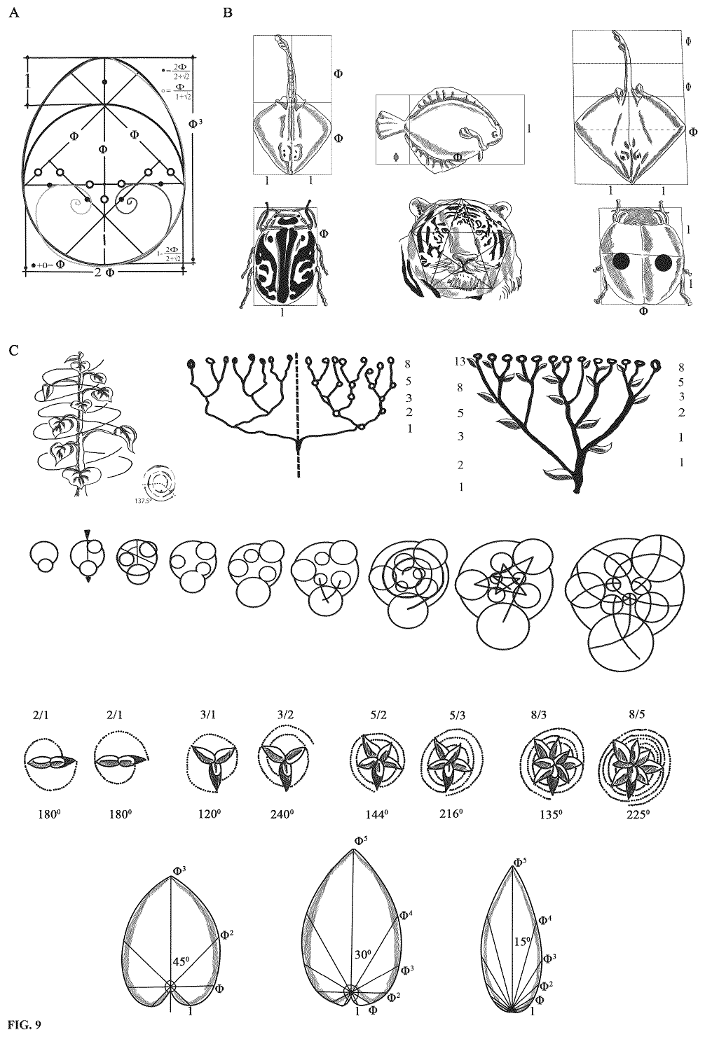

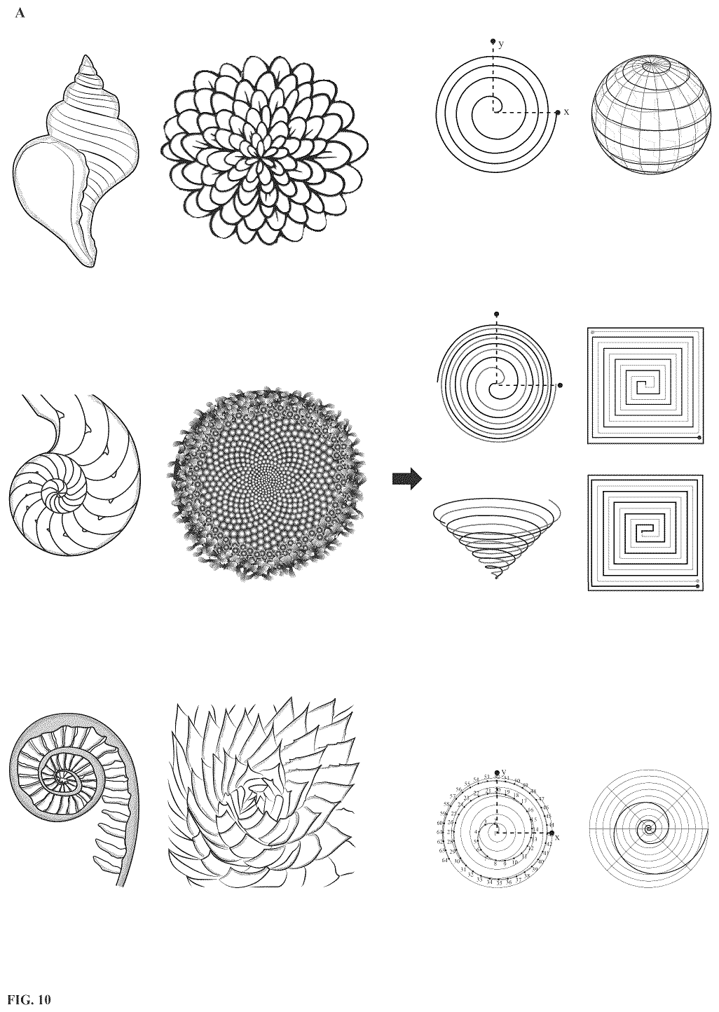

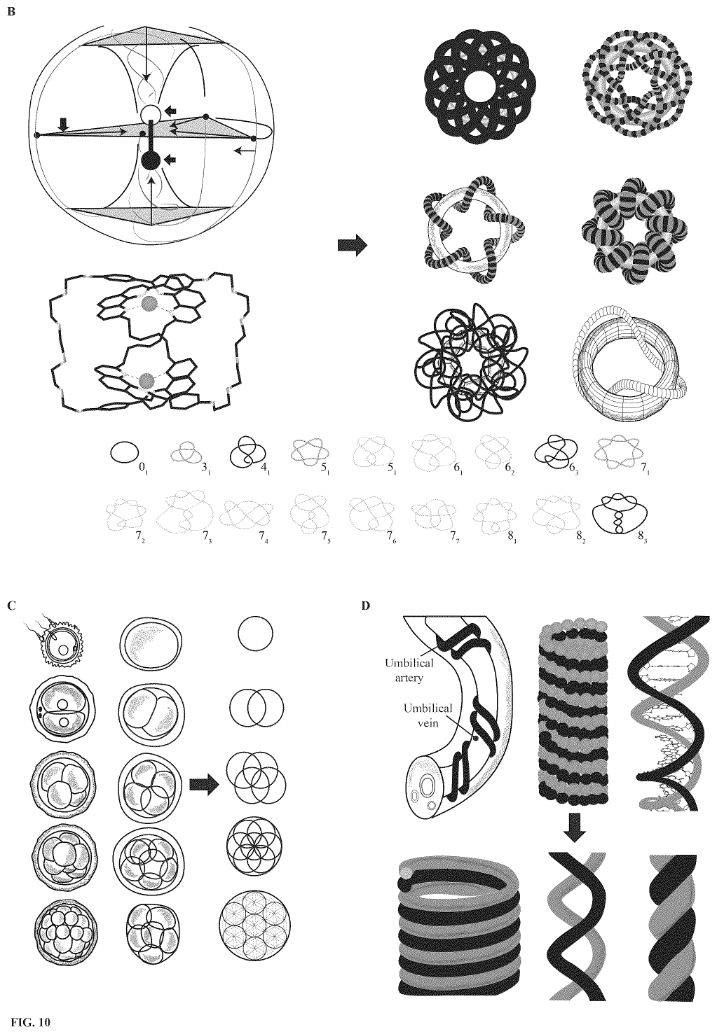

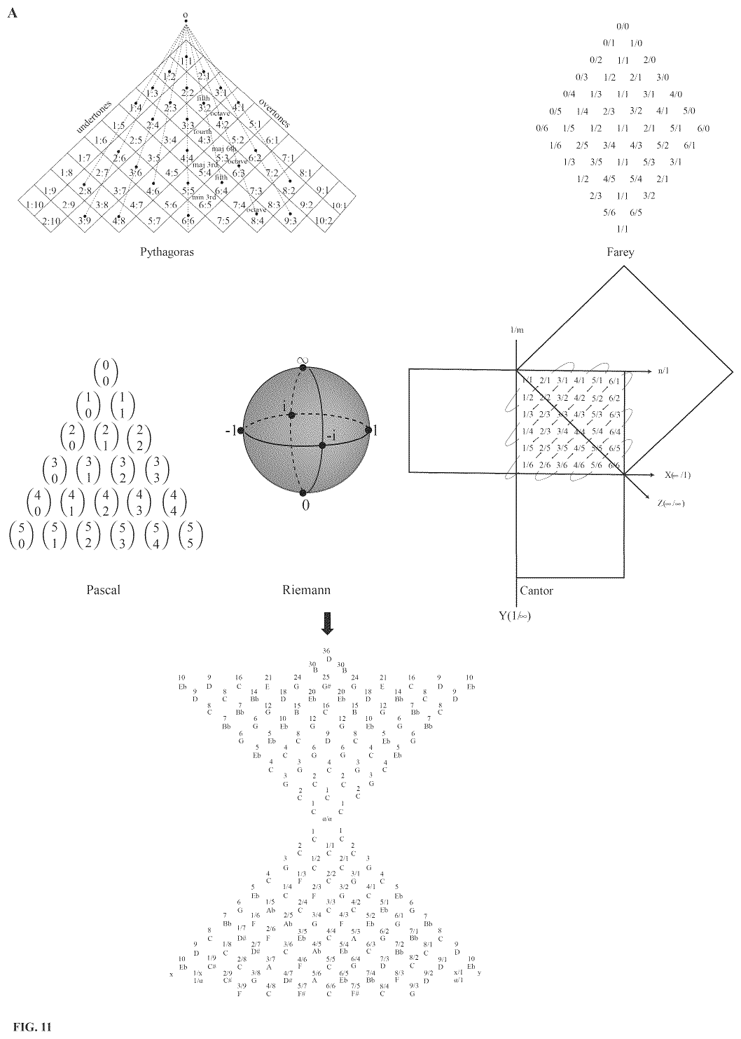

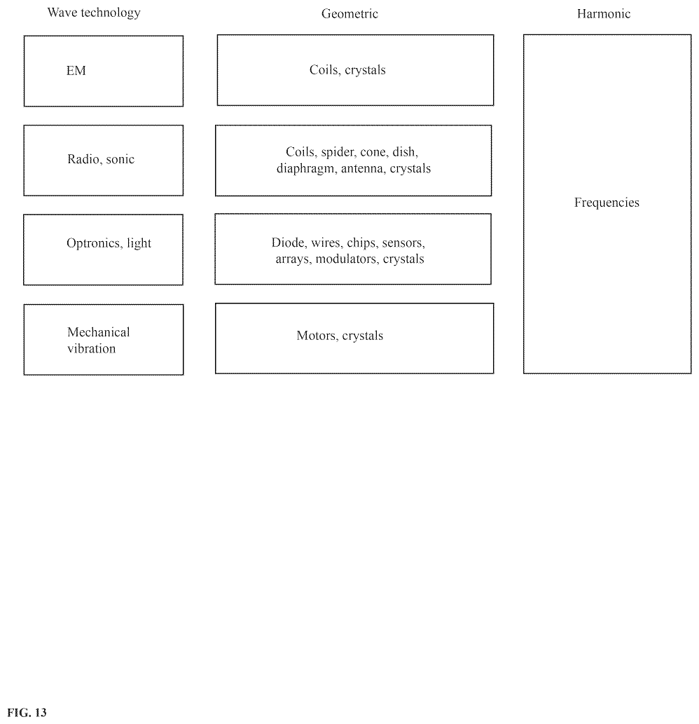

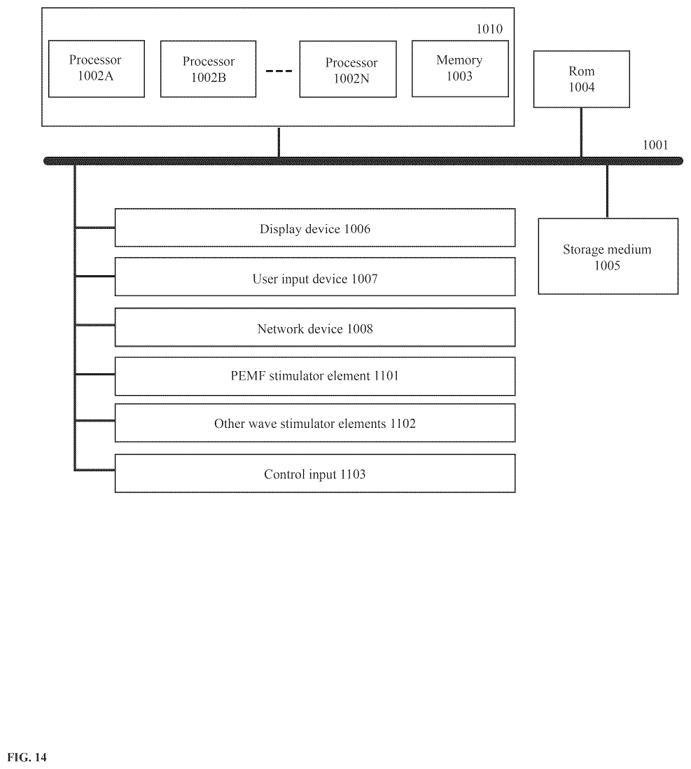

In one exemplary variation, the device comprises a crystalline core that is made of a distinct type of anisotropic, uni/biaxial, negatively/positively-birefringent, transparent crystal, of a specific thickness and number of side facets, which is symmetrically-cut (along the optical axis) and double-terminated (+/−terminals, with specific and different internal angles). The insulated coil, wrapped around the crystalline core, is in the geometric proportions of the DNA, being a coil/supercoil in Φ proportions (FIG. 8B, FIG. 10D, and FIG. 15). The crystalline core and coil may constitute the first part of the device body, which is connected to the second part being the pulse generator (circuit: capacitor charger, capacitor for energy storage, and switch). The pulse generator is engineered to produce novel, mathematically-constructed, harmonic pulse waveform parameters (or frequencies) (FIG. 11), which resonate with different human biological tissues (especially their shared DNA), some of which are specific for stimulating cellular rejuvenation (through “standing waves” generation), while others specific for sexual stimulation. The invention's novel geometric construction, of the crystalline core and coil, ensures a more robust and focused PEMF field and beam, through the generation and maximization of radial polarization (via the “quadratic electro-optic effect”, “photoelectric effect”, “Pockels effect”, “Malus' law”, and aligning the inherent orientation of the crystal lattice's electrical vectors with the direction of the PEMF wave's electric vector component), circular polarization (via the “magneto-optic Faraday effect”), and “second-harmonic generation” or “frequency doubling” (via the specific selection and geometric construction of the crystalline core). The control system may be configured to dynamically control the PEMF stimulation elements or have preset modes of stimulation. The control inputs can be based on received user inputs, preset modes, both, may additionally or alternatively include sensor inputs, and/or be automated to indicate how to vary one or more parameters of stimulation and/or enable/disable one or more modes. The user interface may include physical device inputs (e.g., buttons, switches, dials, and the like) and/or software control inputs. The power system may be battery-powered or alternatively draw power from a power outlet or any other suitable source.

In one exemplary variation, the device comprises the above PEMF variation (wave stimulation without physical touch), as well as, a third device body part with mechanical vibration motors (wave stimulation with physical touch). The motors are distributed within the Trinity Celtic knot engineering design (FIG. 10C and FIG. 15), which is a novel geometric construction that is aligned with the anatomy of the female sex organ (zone 1: glans clitoris and clitoral shaft/body, zone 2: bulbs and crura, and zone 3: vaginal opening, Bartholin glands, perineal sponge, and G-spot), to distribute and maximize the sexual/gynecological stimulation, and thus the incurred benefits by a vulva and vagina-bodied user. The device body is housed within medical grade silicone. The central area of the device's Trinity Celtic knot design may have a novel crystalline finger grip construction, for vibration amplification and ergonomic use by a user (FIG. 15). The vibration of the motors is harmonic (mathematically-constructed to align with a vulva and vagina-bodied user's “sexual response cycle” biology). The control system may be configured to dynamically control the motor stimulation elements or have preset modes of stimulation. The control inputs can be based on received user inputs, preset modes, both, may additionally or alternatively include sensor inputs, and/or be automated to indicate how to vary one or more parameters of stimulation and/or enable/disable one or more modes. The user interface may include physical device inputs (e.g., buttons, switches, dials, and the like) and/or software control inputs. The power system may be battery-powered or alternatively draw power from a power outlet or any other suitable source.

### Form Factor Overview: Handheld, Wearable, Flexible, Rigid

In terms of the form factor, the systems and methods may use a variety of form factors for the device. As a list of exemplary form factors, the system and method may be implemented in connection with a handheld device to be manipulated by a user, a flexible device that includes at least some compliant elements, a set of devices that may operate cooperatively, skin-adhesive patches or a wearable device that can be connected to a user, a clothing-integrated device (e.g., integrated into underwear or the like), and/or other form factors. Herein, the exemplary form factor of a handheld device is used but the system and method are not limited to only that exemplary form factor.

Additionally, the components of the system and method may be integrated into a single device or separated into distinct elements. In one variation, the system and method may be implemented with a small form-factor that is a low-powered, fully-integrated, and handheld. In another variation, a power system may be housed within a separate box and with a wired connection to the handheld device.

In one embodiment, the device comprises a main body, with the main body forming the overall shape of the device. Various components may be embedded, covering, or otherwise attached to the main body. In one variation, the various stimulation elements form the main body. The main body may also be made in part of a semi-flexible material, such as silicon.

In one embodiment, the main body is shaped to conform to or otherwise engage with the shape of sex organs. The main body may feature local density variations to facilitate the application of greater pressure against certain portions of the sex organs and less pressure against other portions. The main body may comprise a handle configured to be held by a human hand. The handle may feature flexible chirality for left and right handed persons, or may have a dedicated chirality. The handle may feature directional chirality for a designated hand-wrist line, or may feature bidirectionality so that the handle may be held via opposing hand-wrist lines.

### Benefits

The system and method may provide a number of potential “sexual wellness” benefits. The system and method are not limited to always providing such benefits, and are presented only as exemplary representations, for how the system and method may be put to use. The list of benefits is not intended to be exhaustive, and other benefits may additionally or alternatively exist.

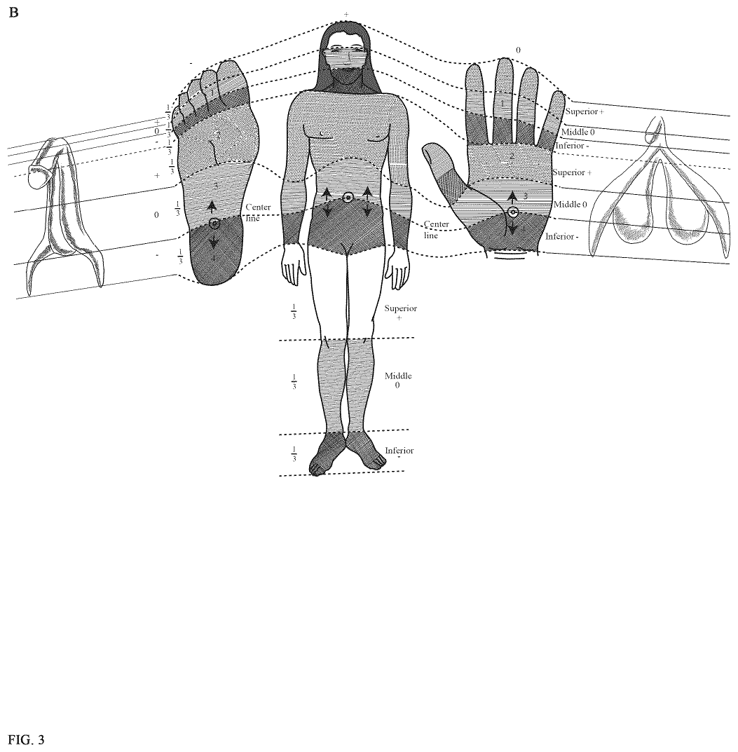

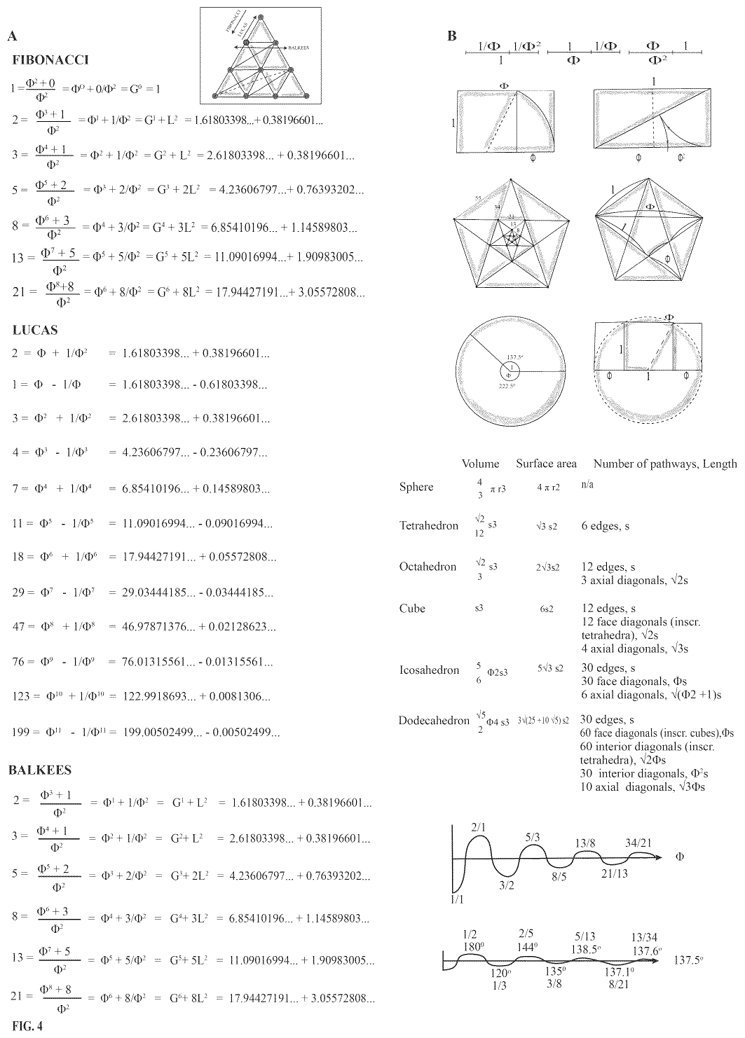

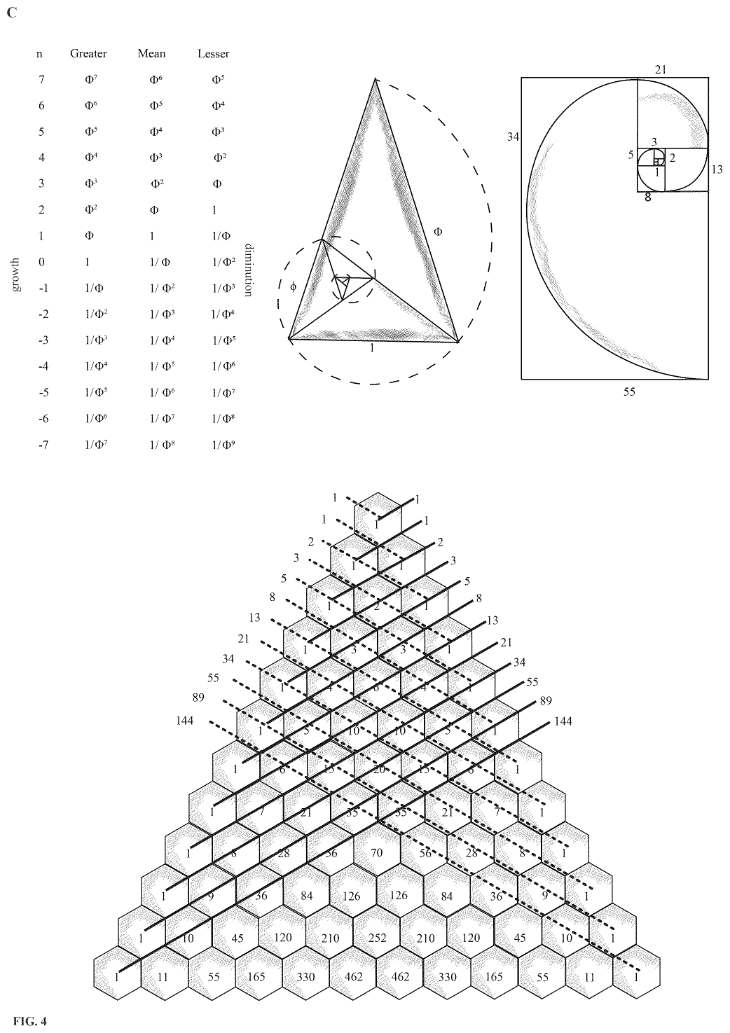

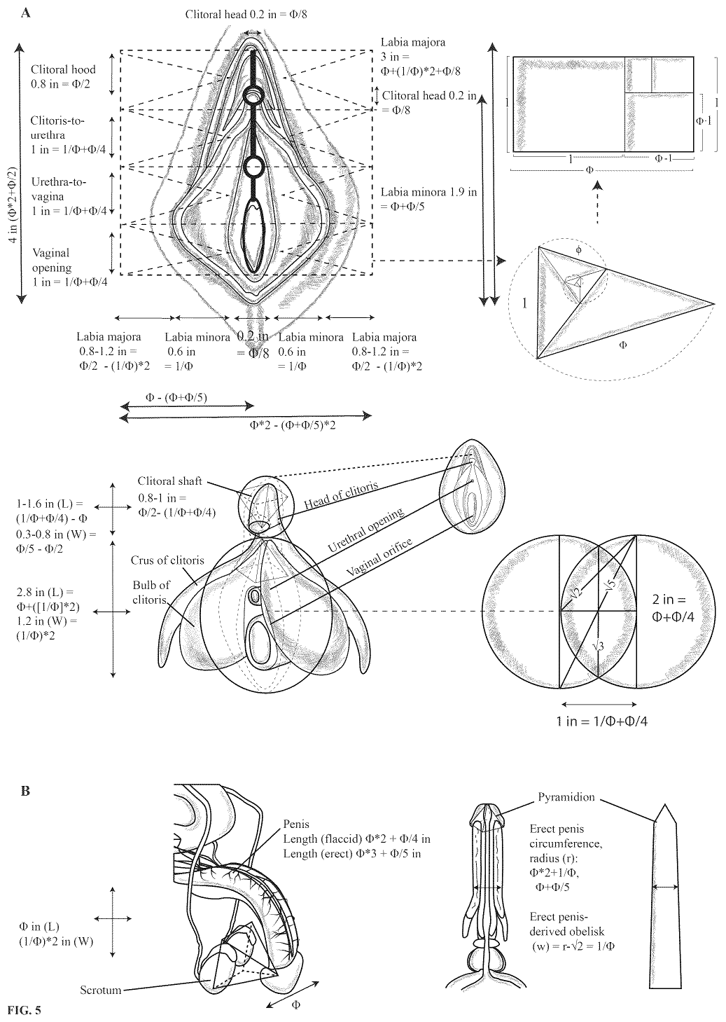

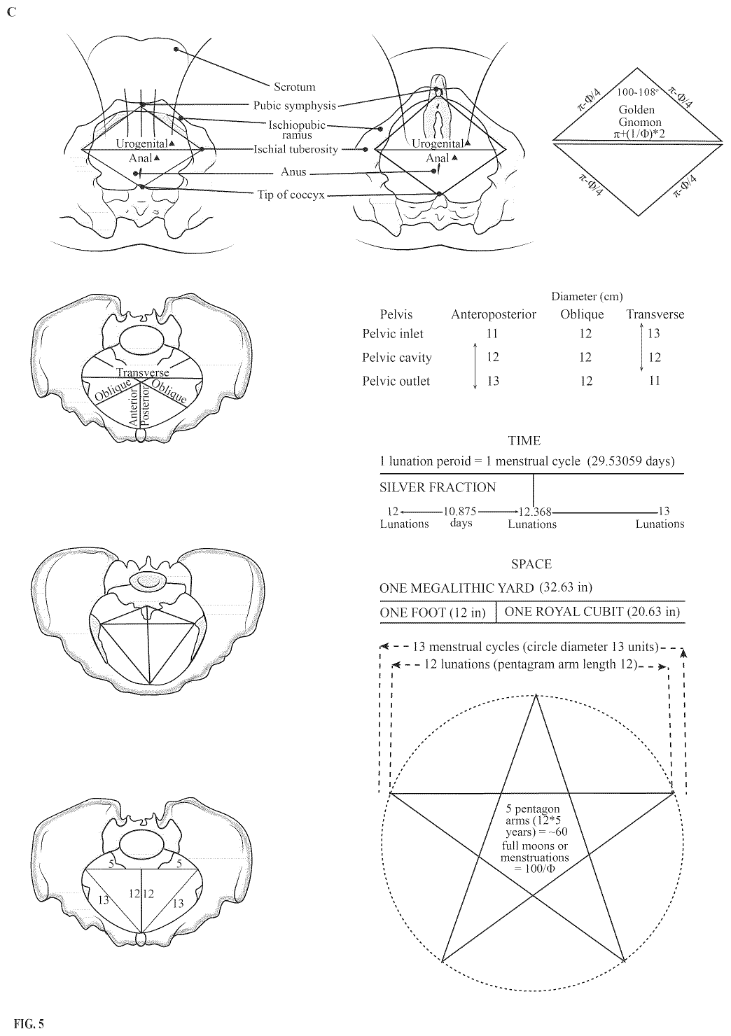

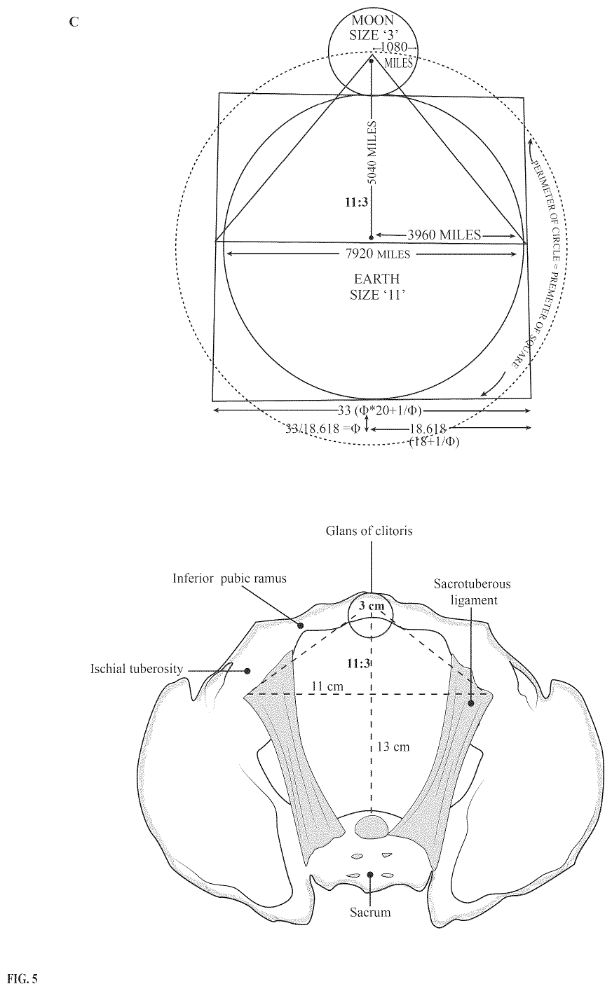

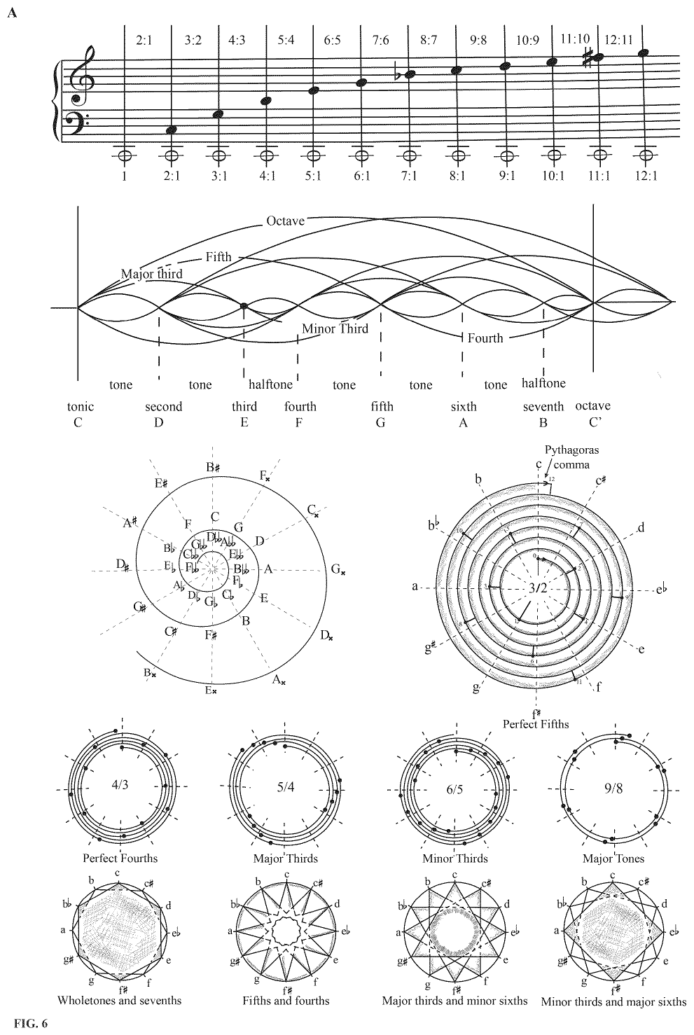

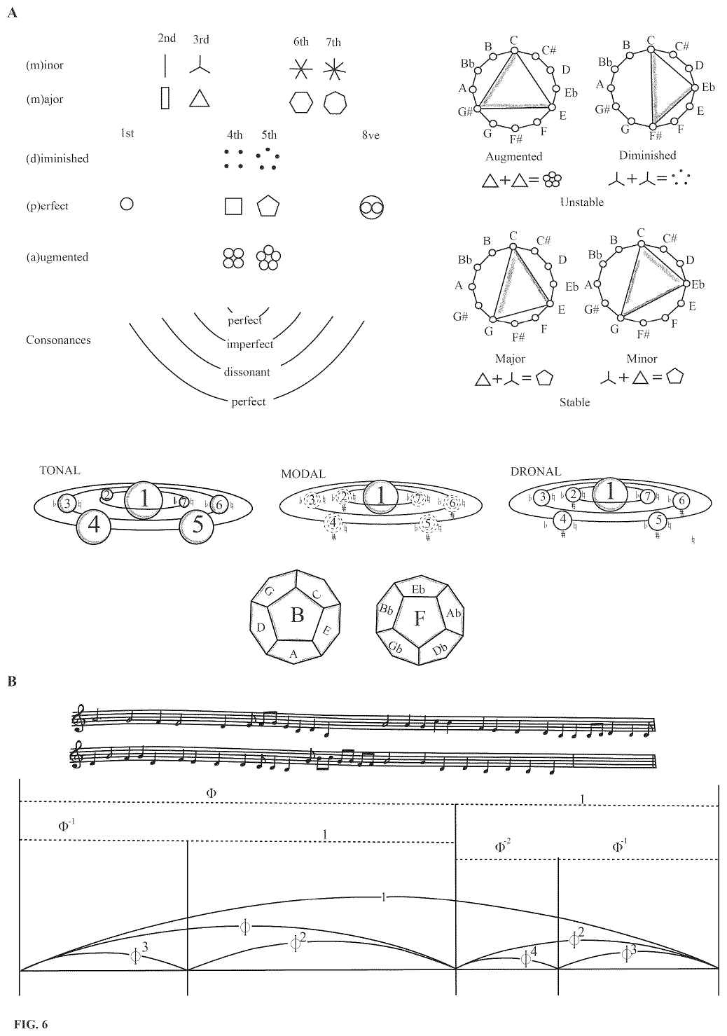

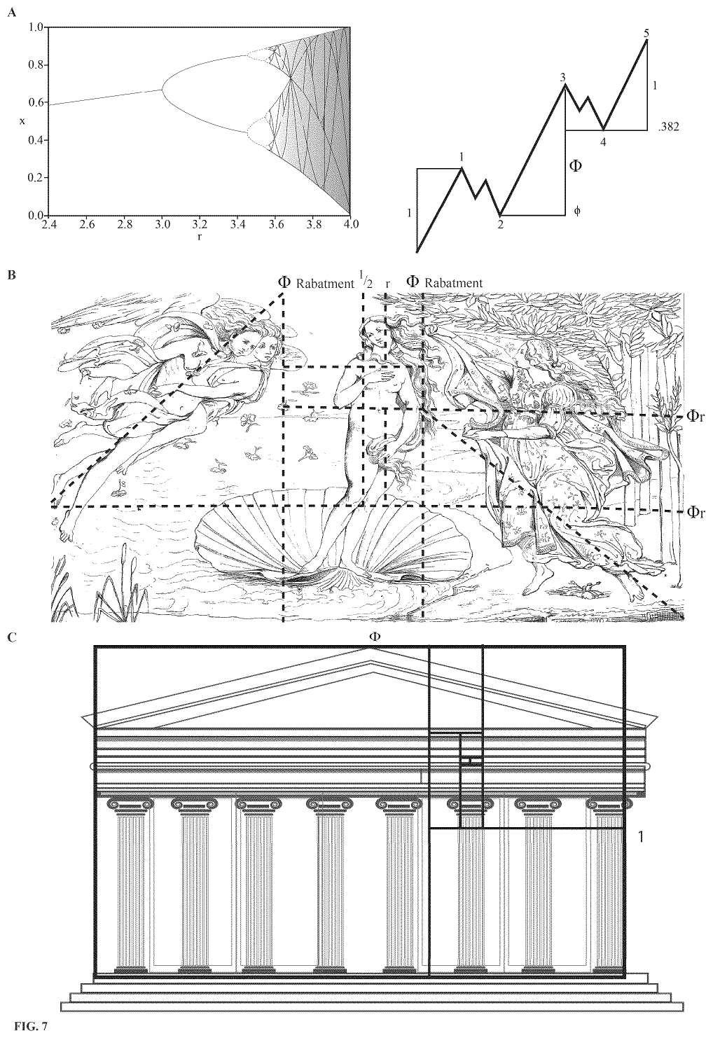

As one potential benefit, the systems and methods may provide superior sexual pleasure stimulation (over existing sexual pleasure stimulation devices). The systems and methods may enable peripheral sexual nerve stimulation within a consumer device. The systems and methods may also enable enhanced targeting of desired regions of the body. At the same time, the systems and methods may provide stimulation to more regions of interest. Different nerve regions of interest (e.g., pudendal n., cavernous ns., G-spot ns., pelvic, vagus, and/or hypogastric branches, FIG. 3A) may be targeted using adaptive targeting, through controlling the PEMF and/or other wave stimulation, and/or the physical ergonomics of the device. In variations designed for vulva and vagina-bodied individuals, this may result in more parts of a clitoris and/or the lower part and front wall of a vagina being targeted and stimulated, causing more nerve stimulation, neural entrainment, muscle contraction, and/or nitric oxide release (i.e., increased blood circulation to the region and/or erection). To a user, this can be experienced as deeper and more therapeutic orgasmic responses, with gynecological health benefits, thus improving their sexual and wellness experience.

As another potential benefit, the systems and methods may employ the PEMF and/or other wave stimulation technology to provide a non-invasive sexual pleasure stimulation approach that is both safe and effective. This may mean that the systems and methods can provide a more pleasurable user experience as a non-invasive tool.

As another potential benefit, the systems and methods may be adaptable to a variety of body types. The physical and dynamic PEMF and/or other wave stimulation design of the systems and methods can enable the device to flexibly provide a pleasurable experience to a wide variety of body types.

The systems and methods may enable a number of potential “gynecological health” benefits. As one gynecological benefit, the systems and methods may simulate the cavernous nerves that may result in vaginal lubrication, congestion, and lengthening, as well as, increased blood flow and/or clitoral erection (through the release of nitric oxide from nerve endings and the endothelium).

As another gynecological benefit, the systems and methods may promote a temperature inside the vagina (e.g., 40-50° C.) that may result in improvements in vaginal elasticity (tightness) and lubrication (moisture). These improvements are mediated through the temperature's effect on collagen synthesis in the vagina, fibroblast stimulation, collagen contraction, neocollagenesis, elastogenesis, vascularization, and/or growth factor production.

As another gynecological benefit, the systems and methods may help with enhancing cellular rejuvenation, reducing inflammation, increasing blood circulation, strengthening the pelvic floor muscles that are essential for childbirth and prevention of pelvic organ prolapse and/or urinary incontinence, and improving sexual functioning (i.e., increased desire, arousal, lubrication, orgasm, satisfaction, and reduced pain/discomfort). Around 75% of women (over 65 years) suffer from urinary incontinence (due to weak pelvic floor muscles from aging, entering menopause, and giving multiple vaginal births). At least a ⅓ of women experience vaginal prolapse (due to weak pelvic floor muscles).

As another gynecological benefit, the systems and methods may stimulate the scrotum-and-penis bodied individual G-spot (or P-spot), whose stimulation can produce powerful orgasms, and contribute to generating thinner seminal fluid from the prostate (for better sperm motility, stability, and energy), thus improving fertility and conception.

As another gynecological benefit, the systems and methods may help with improving and/or treating BPH, ED, and PE. At least 80% of men (over 70 years) suffer from benign prostatic hyperplasia (BPH). Roughly 52% of men experience one form of erectile dysfunction (ED). Around 30-40% of men experience premature ejaculation (PE).

As another gynecological benefit, the systems and methods may help with improving and/or treating STDs. Over 1 million cases of sexually-transmitted diseases (STDs)/day occur worldwide, 374 million cases/year, according to the World Health Organization (WHO).

The systems and methods may enable a number of potential “holistic health” benefits. As one holistic health benefit: geometric and harmonic wave-engineering designs, underpin tissue formation, through “standing wave” formation, which conveys the rejuvenative effect of PEMF (in enhancing the biochemical signaling pathways, including building essential proteins that are reduced and/or lost with aging and disease). Standing waves, which form matter, occur only with geometric PEMF and harmonic frequencies.

As another holistic health benefit: geometric and harmonic wave-engineering designs, underpin biochemical healing, through the “Schrödinger cat state”. Researchers from Berlin, Oxford, and Bristol Universities found that a geometric magnetic field applied to a system, transforms it into a new quantum state called “quantum critical” with a fractal pattern, whereby the system reaches a quantum uncertain (i.e., Schrödinger cat state). Furthermore, by tuning the system and introducing more quantum uncertainty with EM, the chain of atoms acts like a nanoscale guitar string, whereby the spins magnetically-resonate, and can be influenced to enter a healing state.

As another holistic health benefit: geometric and harmonic wave-engineering designs, underpin the human body's transformation into a photoelectrochemical cell, through the absorption of EM and generation of electrical current/voltage (in what's known as the “photovoltaic effect”, “photoelectric effect”, “photoconductivity”, and “Ballistic conduction”). This underpins biochemical healing through replenishing the often deficient cellular voltage (especially with aging and disease).

As another holistic health benefit: geometric and harmonic wave-engineering designs, underpin the improvement of several diseases that are incurable and/or difficult to cure. As one example, researchers found that wave/light therapy (e.g., EM and/or other waves, FIG. 13) stimulated the activity of microglial cells, which are debris-clearing immune cells that influence brain development, spurring changes not only in microglia, but also in the blood vessels, and facilitating the clearance of amyloid plaques that are associated with Alzheimer's and other protein-based diseases. The effect was especially notable in the visual cortex, auditory cortex, and hippocampus of the brain. Even more profound when light and sound (i.e., 2 wave therapies, FIG. 13) were combined.

As another holistic health benefit: geometric and harmonic wave-engineering designs, underpin the improvement of water structure; functionality; sanitation; and drinking-safety, through wave-water interaction. As one example, researchers found that applying the 432 Hz frequency to water, lead to significant reduction in spectral variations and increased water stability. The change of absorbance at water absorbance bands was specific for each frequency. Given that the Earth and human body are made of at least 70% water, and most food sources have water, as well as, having over 2 billion people that live in water-stressed countries (as of 2021, according to the WHO) and acquire dirty water-transmitted diseases (e.g parasitic worms, cholera, diarrhea, dysentery, hepatitis A, typhoid, dengue fever, and polio), geometric and harmonic wave-engineering can become a contributing water sanitation tool. Additionally, out of every 100 patients in acute-care hospitals, 7 patients in high-income countries, and 15 patients in low- and middle-income countries acquire at least one health care-associated infection during their hospital stay due to poor water sanitation. At least 1 million people are estimated to die each year from diarrhea (as a result of unsafe drinking-water). In 2010, the UN General Assembly explicitly recognized access to clean water and sanitation as a “human right”. As another example, researchers from Standard university are using wave therapy to curb mosquito-borne diseases. This invention can contribute to improving the water structure and sanitation for users, which is currently known as “hydrotherapy”.

As another holistic health benefit: geometric and harmonic wave-engineering designs, underpin the reduction of pain, anxiety, and depression levels. As one example, researchers from Harvard Medical School found that harmonic wave therapy (or vibrational medicine) reduces stress levels, improves immune system function, and enhances overall health.

As another holistic health benefit: geometric and harmonic wave-engineering designs promote gamma brain-wave activity, through brain-wave synchronization. Researchers found that exposure to waves at 40 Hz gamma oscillations (25-80 Hz), improves conscious cognition, memory formation, hippocampal memory processing, and functional connectivity in several brain networks. This is called “optogenetics”: noninvasive sensory stimulation to encourage brain neurons to move and synchronize their firing patterns again.

As other potential benefits, the systems and methods may enable enhanced control. The systems and methods may provide preset modes of stimulation that a user can use as is, or modify to their liking. The systems and methods may also provide various control inputs to a user, which enable intuitive and effective ways of enhancing the usage of the holistic health and sexual stimulation device. The device can manage intuitive user-control while being safe. Additionally, the systems and methods may enable smart features where the device can learn how to adapt its operation or provide automated and/or personalized operations.

### System

A system for a PEMF and/or other wave stimulators, aimed at holistic health and sexual pleasure, may include a device body, a set of PEMF stimulation elements around a crystalline core and/or other wave stimulation elements in association with crystals, and a power and control input.

The system can include a power system, a control system, and/or other elements. In some variations, the system may include thermodynamic elements: active and/or passive cooling systems (e.g., liquid coolant, heat sinking). The system and method may include additional components, such as other features for holistic health and sexual stimulation, including but not limited to tactile or haptic stimulators, voice-assistant technology, biofeedback technology, sensor technology, and/or the like.

### Device Body

The device body functions as the structural body of the device. In general, the device body may include the PEMF stimulation elements, other wave stimulation elements, a crystalline lattice, thermodynamic elements, a power system, control inputs, and/or other elements.

In general, the device body will be shaped to promote a particular relative orientation of the PEMF and/or other wave stimulation elements during usage, for functionality and/or ergonomic benefits. In one variation, the PEMF and/or other wave stimulation elements may be positioned adjacent to a targeted region of the body. In other variations, the PEMF and/or other wave stimulation elements may be positioned to promote a more general application of wave stimulation. For example, one or more PEMF and/or other wave stimulation elements may be implemented in a device body, with geometric proportions and harmonic frequencies, to target the whole body and/or its surroundings.

The device body may include features to enhance usability by a user. In particular, the device body may include forms or structures that are designed for usage ergonomics (e.g, how the device conforms to the shape of the body during use). In one variation, the system is used for a handheld device thus the device body may include ergonomic structural features for hand manipulation. In another variation, the device body may be a flexible or compliant body. Alternatively, a sub-portion of the device body could be flexible. This may be used to allow the whole device body, or a portion of it, to be flexibly-moved and manipulated during use. In yet another variation, the device body may be designed to be a substantially-rigid or static form. The static form may, nonetheless, use soft or flexible materials for manipulation during use.

In other variations, the device body may be a part of, or integrated with other ways of positioning and/or manipulating the device. For example, the device body may be implemented as part of a wearable patch such that the device can be temporarily adhered to a part of a user's body. In another example, the device may be integrated into a wearable element such as clothes.

In other variations, the device body may be made of various materials. In some variations, the device body may be at least partially made out of a biocompatible material (e.g., biocompatible silicone).

In other variations, the system may include multiple, distinct device bodies. The distinct device bodies may provide stimulation with the plurality of these distinct bodies. Additionally or alternatively, the distinct bodies may be used to separate functionalities. For example, a driver may be integrated within one device body, while PEMF and/or other wave stimulation elements and/or biofeedback elements may be integrated into a second or more device bodies, with a wired and/or wireless connection between the two bodies. The device body with the stimulation components may be formed for handheld use and/or ambient use, while the device body with the driver component may or may not be intended for manual manipulation.

In yet other variations, the system may be incorporated into a form factor where the user engages with the system. For example, the system could be a sit-on or lie-on form factor, where the user could position their body on top of the device. Such a device may not be designed for manual manipulation, though a device may be designed for both forms of use.

Set of PEMF and/or Other Wave Stimulation Elements

The set of PEMF and/or other wave stimulation elements functions to deliver a form of non-invasive wave stimulation (e.g., PEMF, FIG. 13). The system preferably includes at least one PEMF and/or other wave stimulation element. In some variations, the system includes two PEMF and/or other wave stimulation elements. In other variations, the system may include three or more stimulation elements. All of which are geometrically-constructed with harmonic frequencies. Each stimulation element may be designed to target a particular part of a user's body (sexual wellness) and/or the whole body (holistic health). With multiple stimulation elements, the stimulation elements may be geometrically-constructed and relatively-oriented such that positioning of one stimulation element results in a corresponding position of another stimulation element. Other wave elements may include, but are not limited to: crystals, motors, diodes, wires, chips, sensors, arrays, modulators, spiders, cones, dishes, diaphragms, and/or antennas.

### PEMF: Basic Components

The insulated PEMF stimulation element is housed around a crystalline core forming the device body or a part of it. A PEMF stimulation element preferably includes at least a pulse generator and a coil (FIG. 10 and FIG. 13). The pulse generator functions as a circuit (i.e., electronic control device), which can convert an electrical power input and control inputs, into an electrical signal fed through the coils, to create a pulsed electromagnetic field. A control system may be used for the direct control of a PEMF stimulation element.

### PEMF: Coils and Crystalline Core

The coils are preferably integrated into the device body according to intended use. In some variations, the coils may be positioned into regions of the device body such that they provide general stimulation. In other variations, the coils may be integrated into particular portions of the device body such that they provide anatomically-specific stimulation during intended use. Coil design can be configured per different stimulation objectives. A coil may be designed for superficial targeting only. Another coil may be designed for deeper targeting only. Another coil may be designed for varying degrees of both. Another coil may be designed for whole body stimulation. Yet another design might be targeted for whole space stimulation with more than 1 person involved. Such design features may be customized depending on the objectives of the implementation.

The coils have geometric constructions configured according to various factors, such as: desired focality of stimulation, depth of stimulation, energy efficiency (e.g., energy recovery, reduced capacitive losses in circuit elements, reduced eddy current losses in the coil winding conductors, reduced losses in the ferromagnetic core [if any], reduced internal resistance and overheating in the coil and other circuit components), how the direction of incident PEMF impacts the refractive index of a crystal, how the geometric combination of a crystal and PEMF impacts the generation of the “Kerr effect” and “Pockels effect”, how the geometric combination of a crystal and PEMF impacts the “photoelectric effect”, how the geometric combination of a crystal and PEMF impacts the generation of “standing waves”, how the geometric parameters of the coil and core crystal impact the robustness and purity of the PEMF fields. The parameters of the coils and crystals may vary in different implementations. In some variations, the coil design may be a computationally-optimized winding pattern. In other variations, the coils may be coupled with ferromagnetic material (with high magnetic permeability) to focus the magnetic field energy towards the target tissue.

There may be a variety of approaches to configuring coil shape, size, placement, and/or orientation within the system. All of which are mathematically and geometrically-constructed for stimulation efficacy and power benefits. Different variations may have different device size, stimulation efficiency, energy efficiency, and/or packaging benefits. The coils may be helicoidal configured according to various factors (e.g., handedness (chirality), dilation symmetry, number of turns, turn spacing (minor and major grooves, if double helix), knotting patterns with electrical charge differences, packing density, radius of the helical cylinder, helical pitch angle, helical tube radius, coil diameter, coil It-based repetition length that defines the slope of the curve, and number of winds wrapped around a torus of major radius R and minor radius r with t based on the number of sets of xyz coordinates).

There may be a variety of approaches to configuring factor size and shape. All of which are mathematically and geometrically-constructed for stimulation efficacy, power, and ergonomic benefits. The system may include anatomically-external, internal, and/or both form factors.

The PEMF coil crystalline core and/or auxiliary structure may be configured according to various factors (e.g., how the thickness of the crystalline lattice impacts its refractive index, how the dielectric constant of a crystal impacts the speed at which incident PEMF propagates through it, how the refractive index of a crystal varies with the direction of incident PEMF, how the optical transparency of a crystal impacts its dielectric permittivity tensor and thus ability to become circularly-birefringent, how the geometric and harmonic combination of a crystal and PEMF generates the “Kerr effect” and “Pockels effect”, how the birefringence of a crystal plays an important role in “second-harmonic generation” or frequency doubling, how the geometric and harmonic combination of a crystal and PEMF optimizes the “photoelectric effect”, how the geometric and harmonic combination of a crystal and PEMF generates “standing waves”, how the geometric parameters of the crystal and coil impact the generation of pure and robust PEMF fields).

### PEMF: Pulse Generator and Control Inputs

The PEMF driver functions to translate control input and electrical power into a signal to drive the coils (i.e., simulator, pulse generator). The PEMF driver (with its capacitor charger, capacitor for energy storage, and high-power switch) may be configured differently to control various parameters of energy delivery to the coils. In particular, the driver may control the voltage/current, current direction/reversal, pulse repetition frequency, E-field waveforms; pulse shape; polarity; and/or amplitude, and/or other properties. A driver circuit may be used for each coil. Alternatively, a driver circuit may be used to drive multiple coils.

In some variations, the driver may be integrated into the same device body as the coils. In other variations, some or all of the driver may be integrated into a separate device body. When the driver is integrated into a separate device body, a wired connection to the device body with the coils may be used.

A driver circuit could be, but is not limited, to any of the following topologies: monophasic, biphasic, rectangular, H-bridge, or a combination thereof.

The control inputs direct the function of the device. In some variations, the control input could be based on received user inputs. In another variation, the control input could be based on preset modes. In other variations, the control input could be based on received user inputs and preset modes. In yet other variations, the control input may be automated using a control system with control inputs, which supply signals to indicate how to vary one or more parameters of stimulation, and/or enable/disable one or more modes as different preset forms of stimulation.

The control inputs may be used in managing various control vectors, such as: voltage (e.g., ability to drive voltage up and down), repetition frequency, pulse waveform parameters, polarity reversal, and/or other properties. The control inputs may be used to control variables of significance for a user's sexual wellness and/or holistic health experience (e.g., stimulation intensity, by modifying voltage and/or current, to have an intensity range for the varying stimulation thresholds in the same individual over time, and the varying stimulation thresholds among different individuals). The control inputs may additionally provide independent and/or coupled control of the set of PEMF and/or other wave stimulation elements.

The control inputs may be used to alter the nature of stimulation of a target (e.g., changing focus, altering depth between superficial and deep, redirecting/steering).

The control inputs may include one or more user interface inputs. The user interface inputs may include physical device inputs. The physical device inputs could include user interface elements presented on the device body. For example, the physical device inputs could include buttons, switches, dials, and the like.

The control inputs may include one or more additional or alternative interface inputs. The user interface may include software control inputs. The software control inputs may be communicated from an application or other suitable type of software user interface. For example, a user may download an app on their personal computing device and use an interface within the app to control the device. The user may also give verbal commands to the device that has a voice-assisted technology/software and, in turn, the device responds and communicates audibly (for a hands-off user experience). The user interface inputs can directly impact one or more variables. In one variation, the device may expose a set of user interface inputs to individually control stimulation intensity and/or pulse frequency. In another variation, the user interface inputs may be used to select a mode that comes with pre-configured variable settings (e.g., the user interface inputs may be used to select a gentle and build-up stimulation mode or an intense and quick stimulation mode. A wide variety of modes may be offered to a user).

The control inputs may additionally or alternatively include sensor inputs. The sensor inputs could include an inertial measurement unit (IMU), accelerometer, gyroscope, magnetometer, temperature sensor, pressure sensor, biometric sensor (e.g., heart rate sensor, respiratory sensor, body temperature), contact sensors, and/or other suitable sensors. These may provide sensor data that can be used to interpret the state of a user. The sensor data may be used to augment the control signals of a PEMF and/or other wave stimulation elements. For example, rhythmic motion of the device may be detected and used to synchronize variations of the PEMF stimulation signals. In some cases, external data sources may also be used to alter the control inputs. Examples of external data sources may include data from a period tracking app, a health monitor, a mental health check app, and/or other suitable data.

In one embodiment, the device may comprise a control module. The control module may be configured to receive commands from a user of the device. In one version, the control module may comprise a controller and a receiver, with the controller configured to be held by a user while the main body is held by the user with a different hand or by a different user altogether. In this version, the controller is physically separate from the main body, which in turn comprises a receiver for wireless or wired communication with the controller. In one variation, the controller is a dedicated device; in another variation, the controller is a conventional mobile device.

In an alternative or additional embodiment, the controller may be embedded or otherwise attached to extend from the main body, so that a user can instructionally as well as physically operate the device with only a single hand.

The controller may be configured to simultaneously operate all stimulation elements, or operate one or more in isolation.

In one embodiment, the crystal comprising the crystal core is selected and cut in order to obtain a crystal configuration having a crystallographic structure and shape to resonate best with biological tissue. The crystallographic structure may include the arrangement of molecules in the crystalline material. Other crystallographic features for which the crystal may be configured include its symmetry, optical transparency, and cleavage.

In one embodiment, the control module comprises a set of sensors, input devices, a readable memory, and a processor, with the processor programmed to select a preset mode based on feedback received from the sensors and/or the input devices, with present modes being saved to the readable memory. The preset modes may designate stimulation elements, electronic parameters thereof, and during for which a given preset mode operates.

### Control and Power System

A control system can function to facilitate how to drive a stimulation element. The control system preferably determines how to control stimulation based on various factors. In a more direct control system, control inputs may be translated directly to control the corresponding variables of PEMF and/or other wave stimulation elements. The control system may alternatively be more dynamic to modify control of the stimulation variables, based on various properties, such as: control inputs, sensor data, user data, battery power, and/or the like.

The control system may be configured to dynamically control the PEMF and/or other wave stimulation elements, to enable different stimulation experiences. In one variation with PEMF stimulation elements, they may be controlled to selectively stimulate the clitoral head or clitoral complex (i.e., with each stimulation element targeting one region at a time). In another variation, stimulation elements may be stimulating both regions at the same time or stimulating them in some coordinated sequence (i.e., the control system may be used to start stimulation at the head and then transition to primarily stimulate the complex, or alternatively start at the complex and transition to the head). In yet another variation, stimulation elements may be stimulating the whole body and/or a group of bodies with different frequencies.

The power system functions to provide power to operate the system. The power system may be battery-powered. A battery may be integrated into the device such that it can function as an all-in-one device. A battery may alternatively be integrated into an external device body that can be tethered to the main stimulation device body. The power system may alternatively draw power from a power outlet or any other suitable source. The power system may be used to power stimulation, computation, and/or other operations of the device.

### Method

The method for a PEMF holistic health and sexual pleasure device can include providing a PEMF and/or other wave stimulation elements and then controlling the PEMF and/or other wave stimulation elements for tissue stimulation (whether it was sex organ cellular stimulation or whole body cellular rejuvenation). In one variation, the method might involve controlling the PEMF and/or other wave stimulation elements according to supplied user inputs, which functions to adjust stimulation according to user inputs. In another variation, the method might involve controlling the PEMF and/or other wave stimulation elements according to preset modes of stimulation. Additionally, the method may include learning stimulation patterns and applying subsequent stimulation, based in part on learned stimulation patterns. For example, preferences of a user may be learned, and then used to better provide stimulation for a user. The method may also include incorporating sensor data, biofeedback data, apps data, and/or responding to a user's verbal or gesture commands.

### System Architecture

The systems and methods of the embodiments can be embodied and/or implemented, at least in part as machine configured, to receive a computer-readable medium and storing computer-readable instructions. The instructions can be executed by computer-executable components integrated with the application, applet, host, server, network, website, communication service, communication interface, hardware, firmware, or software elements of a user computer or mobile device, wristband, smartphone, or any suitable combination thereof. Other systems and methods of the embodiment can be embodied and/or implemented, at least in part as machine configured, to receive a computer-readable medium and storing computer-readable instructions. The instructions can be executed by computer-executable components integrated with apparatuses and networks of the type described above. The computer-readable medium can be stored on any suitable computer readable media, such as: RAMs, ROMs, flash memory, EEPROMs, optical devices (CD or DVD), hard drives, floppy drives, or any suitable device. The computer-executable component can be a processor, but any suitable dedicated hardware device can alternatively or additionally execute the instructions.

In one variation, a system comprising of one or more computer-readable mediums (e.g., non-transitory computer readable mediums) storing instructions that, when executed by one or more computer processors, cause a computing platform to perform operations comprising those of the system or method described herein, such as: providing a PEMF and/or other wave stimulation device and operating the PEMF and/or other wave stimulation elements.

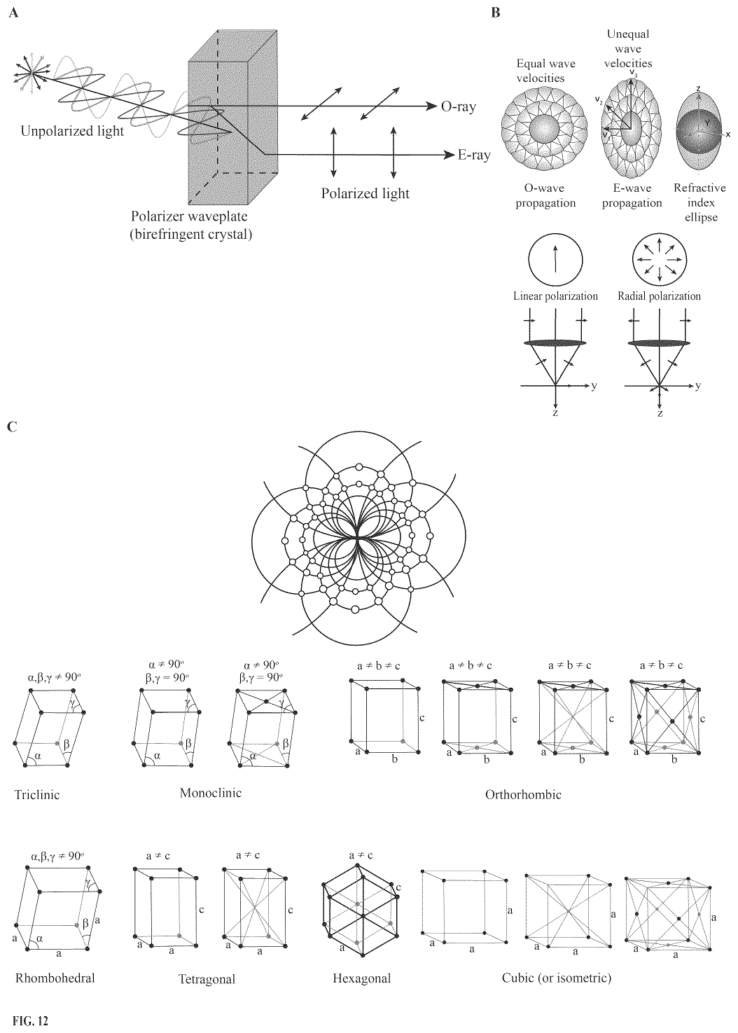

FIG. 14 is an exemplary computer architecture diagram of one implementation of the system. In some implementations, the system is implemented in a plurality of devices in communication, over a communication channel and/or network. In some implementations, the elements of the system are implemented in separate computing devices. In other implementations, two or more of the system elements are implemented in the same devices. The system and portions of the system may be integrated into a computing device that can serve as the system or within the system.

The communication channel 1001 interfaces with the processors 1002A-1002N, the memory (e.g., a random access memory (RAM)) 1003, a read only memory (ROM) 1004, a processor-readable storage medium 1005, a display device 1006, a user input device 1007, and a network device 1008. As shown in FIG. 14, the computer infrastructure may be used in connecting the PEMF stimulator element 1101, other wave stimulation elements 1102, control input 1103, and/or other suitable computing devices.

The processors 1002A-1002N may take many forms, such as: CPUs (Central Processing Units), GPUs (Graphical Processing Units), microprocessors, ML/DL (Machine Learning/Deep Learning) processing units like a Tensor Processing Unit, FPGA (Field Programmable Gate Arrays), custom processors, and/or any suitable type of processor.

The processors 1002A-1002N, and the main memory 1003 (or some sub-combination), can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors that execute the received instructions. In some embodiments, the processing unit is an ASIC (Application-Specific Integrated Circuit). In other embodiments, the processing unit is a SoC (System-on-Chip). In yet other embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (e.g., an operating system, application programs, and device drivers) can be stored in the memory 1003, from the processor-readable storage medium 1005; the ROM 1004; or any other data storage system.

When executed by one or more computer processors, the respective machine-executable instructions may be accessed by at least one of the processors 1002A-1002N (of a processing unit 1010) via the communication channel 1001, and then executed by at least one of the processors 1002A-1002N. Data, databases, data records, or other stored forms of data, created or used by the software programs, can also be stored in the memory 1003, and such data is accessed by at least one of the processors 1002A-1002N, during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid state drive, a ROM, an EEPROM, an electronic circuit, a semiconductor memory device, and the like. The processor-readable storage medium 1005 can include an operating system, software programs, device drivers, and/or other suitable sub-systems or software.

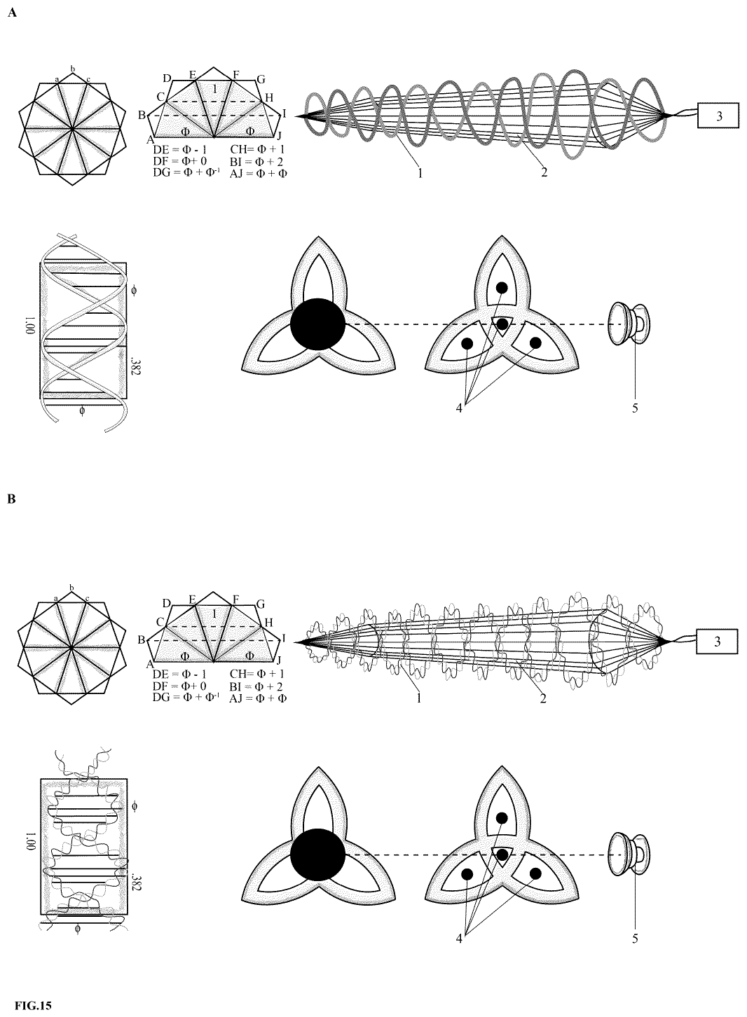

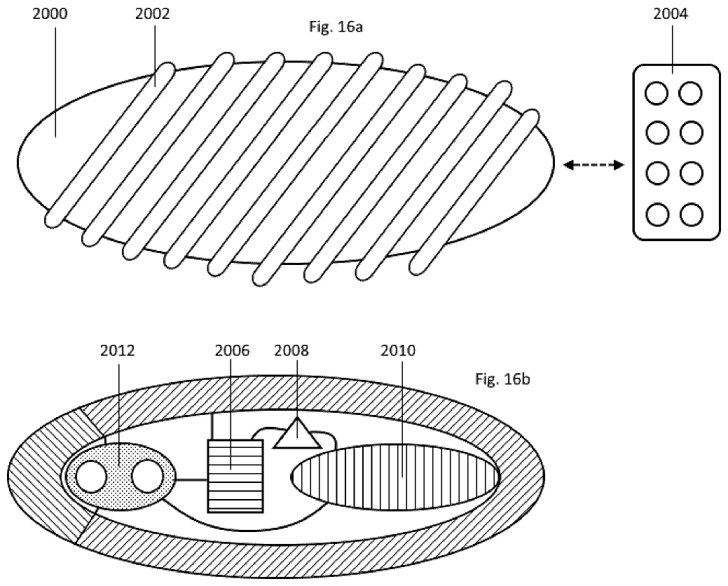

As shown in FIGS. 16a-16b, the device may comprise a main body 2000, which may comprise coils 2002, a power source such as a removable or chargeable battery 2012, a PEMF stimulation element 2006, and a mechanical vibration stimulation element 2010. The coils may be in electronic communication with the PEMF stimulation element, with the latter providing a current for the former. The device may also comprise a control module for manipulating the PEMF stimulation element and mechanical vibration stimulation element. The control module may comprise a controller 2004 which may engage wirelessly with a local receiver 2008, with the latter embedded in or otherwise attached to the main body.

As used herein, first, second, third, etc., are used to characterize and distinguish various elements, components, regions, layers, and/or sections. These elements, components, regions, layers, and/or sections should not be limited by these terms. Use of numerical terms may be used to distinguish one element, component, region, layer, and/or section from another element, component, region, layer, and/or section. Use of such numerical terms does not imply a sequence or order, unless clearly indicated by the context. Such numerical references may be used interchangeably without departing from the teaching of the embodiments and variations herein.

As a person skilled in the art will recognize from the previous detailed description and from the figures and claims, modifications and changes can be made to the embodiments of the invention, without departing from the scope of this invention as defined in the following claims.

1. A personal stimulation device for holistic health and sexual pleasure, comprising:
a. a main body, with the main body being handheld, made in part of a semi-flexible material, and configured for stimulating sex organs;
b. a PEMF stimulation element disposed within or forming in part the main body, with the PEMF stimulation element configured to generate pulsating electromagnetic fields to stimulate the sex organs via the pulsating electromagnetic fields;
c. a control module, with the control module being configured to control the PEMF stimulation element.

2. The personal stimulation device of claim 1, with the control module comprising a handheld controller and receiver, with the handheld controller being in wireless communication with the receiver and the receiver being embedded in the main body and in electronic communication with the PEMF stimulation element.

3. The personal stimulation device of claim 1, additionally comprising a crystalline core, with the crystalline core being disposed within or forming in part the main body and configured to modify the electromagnetic fields generated by the PEMF stimulation element.

4. The personal stimulation device of claim 3, with the PEMF stimulation element being disposed around the crystalline core.

5. The personal stimulation device of claim 3, with the crystalline core having a crystalline structure and cut configured for engagement with the PEMF stimulation element.

6. The personal stimulation device of claim 3, with the PEMF stimulation element comprising an insulated coil.

7. The personal stimulation device of claim 6, with the pulsating electromagnetic fields being formed by an electrical signal passing through the insulated coil.

8. The personal stimulation device of claim 7, with the voltage, current, or pulse waveform parameters of the electrical signal being controlled by the control module.

9. The personal stimulation device of claim 1, additionally comprising a mechanical vibration stimulation element disposed within or forming in part the main body, with the mechanical vibration stimulation element comprising vibration motors configured to stimulate the sex organs.

10. The personal stimulation device of claim 2, with the control module comprising a controller and a receiver, with the controller being configured to control the mechanical vibration stimulation element via the receiver, with the receiver being in electronic communication with the mechanical vibration stimulation element.

11. The personal stimulation device of claim 1, with the control module comprising input devices, with the set of input devices comprising buttons, switches, dials, or a touchscreen interface.

12. The personal stimulation device of claim 1, with the control module comprising a set of sensors, with the set of sensors comprising an accelerometer, gyroscope, temperature sensor, pressure sensor, biometric sensor, or contact sensor.

13. The personal stimulation device of claim 1, with the control module comprising a processor programmed to run preset modes of PEMF stimulation.

14. The personal stimulation device of claim 9, with the control module comprising a processor programmed to run preset modes of mechanical vibration stimulation.

15. The personal stimulation device of claim 1, additionally comprising a power source, with the power source being a battery or an electrical plug.

16. A personal stimulation device for holistic health and sexual pleasure, comprising:
a. a main body, with the main body being handheld, made in part of a semi-flexible material, and configured for sex organ stimulation;
b. a pulse generator disposed within the main body, with the pulse generator configured to generate pulses to stimulate sex organs;
c. a control module, with the control module configured to control the pulse generator.

17. The personal stimulation device of claim 16, with the pulse generator configured to harmonize the pulses with biological tissue of the sex organs, with harmonization between the pulses and the biological tissue achieved via receiving feedback from biofeedback sensors and modifying the pulses based on the feedback.

18. A personal stimulation device for holistic health and sexual pleasure, comprising a main body, a PEMF stimulation element, a crystalline core, and a mechanical vibration stimulation element;
a. with the main body being handheld, made in part of a semi-flexible material, and configured for sex organ stimulation;
b. with the PEMF stimulation element disposed within the main body and at least partially covering the crystalline core, with the PEMF stimulation element configured to generate pulsating electromagnetic fields to stimulate sex organs;
c. with the crystalline core being disposed within the main body and configured to modify the electromagnetic fields generated by the PEMF stimulation element;
d. with the crystalline core having a crystalline structure and cut configured for engagement with the PEMF stimulation element;
e. with the mechanical vibration stimulation element disposed within the main body, with the mechanical vibration stimulation element comprising vibration motors and geometrically configured to stimulate the whole sex organs.

19. The personal stimulation device of claim 18, with the PEMF stimulation element configured to harmonize the pulsating electromagnetic fields with biological tissue of the sex organs.

20. The personal stimulation device of claim 18, with the mechanical vibration stimulation element configured to harmonize the mechanical vibrations with biological tissue of the sex organs.
